(19) United States
(12) Patent Application Publication
(10) Pub. No.: US 2026/0102105 A1
(43) Pub. Date: Apr. 16, 2026

(54) BIOLOGICAL SIGNAL MEASUREMENT SYSTEM, BIOLOGICAL SIGNAL MEASUREMENT APPARATUS, DATA ANALYSIS APPARATUS, BIOLOGICAL SIGNAL PROCESSING METHOD, BIOLOGICAL SIGNAL MEASUREMENT PROGRAM, DATA ANALYSIS PROGRAM

(71) Applicants: Murata Manufacturing Co., Ltd., Nagaokakyo-shi (JP); VIE, INC., Kamakura-shi (JP)

(72) Inventors: Motoyasu NAKAO, Nagaokakyo-shi (JP); Yuji NIHEI, Nagaokakyo-shi (JP); Yusuke YOSHIDA, Nagaokakyo-shi (JP); Yasuhiko IMAMURA, Kamakura-shi (JP); Yasushi NARUSE, Kamakura-shi (JP)

(73) Assignees: Murata Manufacturing Co., Ltd., Nagaokakyo-shi (JP); VIE, INC., Kamakura-shi (JP)

(21) Appl. No.: 19/422,481

(22) Filed: Dec. 17, 2025

Related U.S. Application Data
(63) Continuation of application No. PCT/JP2024/023707, filed on Jul. 1, 2024.

(30) Foreign Application Priority Data
Jul. 3, 2023 (JP) 2023-109426

(51) Int. Cl. A61B 5/38; A61B 5/31

(52) U.S. Cl. CPC A61B 5/38; A61B 5/31

(57) ABSTRACT

A biological signal measurement system includes a biological signal measurement apparatus and a data analysis apparatus. The data analysis apparatus generates a stimulation signal and wirelessly transmits the stimulation signal to the measurement apparatus. The measurement apparatus measures a biological signal of a subject responding to the stimulation. The measurement apparatus acquires a trigger signal corresponding to the stimulation signal, and utilizes a synchronous sampling circuit to synchronize and sample the trigger signal and the biological signal, generating a synchronously sampled data set. The measurement apparatus transmits the synchronously sampled data set to the data analysis apparatus. The data processing apparatus performs data processing on the received biological signal and trigger signal within the synchronously sampled data set to time-align the biological signal with the trigger signal.

### CROSS-REFERENCE TO RELATED APPLICATIONS

This application claims priority to International Application No. PCT/JP2024/023707, filed on Jul. 1, 2024, which claims priority to Japanese Patent Application No. 2023-109426, filed on Jul. 3, 2023, the entire contents of each of which are incorporated herein by reference.

### TECHNICAL FIELD

The present disclosure relates to a system that measures a biological signal of a subject by a sensor that the subject wears.

### BACKGROUND ART

Patent Literature 1 describes a biological signal measurement system. The biological signal measurement system of Patent Literature 1 includes a biological signal measurement apparatus and an information processing apparatus. The biological signal measurement apparatus and the information processing apparatus wirelessly communicate with each other.

The information processing apparatus outputs stimulation for measuring a biological signal. The biological signal measurement apparatus measures the biological signal corresponding to the stimulation and sends to the information processing apparatus.

The biological signal measurement apparatus and the information processing apparatus each include a timing generation portion. The biological signal measurement apparatus and the information processing apparatus, by performing processing at a timing at which each apparatus generates, synchronize the timing of stimulation and the timing of wireless communication.

### CITATION LIST

### Patent Literature

Patent Literature 1: Japanese Unexamined Patent Application Publication No. 2021-183046

### SUMMARY

A biological signal measurement system of the present invention includes a biological signal measurement apparatus and a data analysis apparatus. The biological signal measurement apparatus includes a sensor that measures a biological signal based on reaction of a subject to stimulation, and a first wireless communication portion. The data analysis apparatus includes a data processing portion that performs predetermined data processing to the biological signal, and a second wireless communication portion that performs data communication with the first wireless communication portion. The data analysis apparatus includes a stimulation signal generation portion that generates a stimulation signal for giving stimulation. The biological signal measurement apparatus includes a trigger signal obtaining portion that obtains a trigger signal corresponding to the stimulation signal, and a synchronous sampling portion that synchronizes and samples the trigger signal and the biological signal. The second wireless communication portion sends the stimulation signal to the first wireless communication portion, and the first wireless communication portion receives the stimulation signal from the second wireless communication portion. The first wireless communication portion sends the trigger signal and the biological signal that have been sampled by the synchronous sampling portion to the second wireless communication portion, and the second wireless communication portion receives the trigger signal and the biological signal from the first wireless communication portion. The data processing portion performs the predetermined data processing, based on the received biological signal and trigger signal.

In this configuration, the trigger signal and the biological signal synchronized with the stimulation signal are synchronized and sampled and are sent as a signal pair (or synchronously sampled data set) to the data analysis apparatus. Therefore, even when wireless communication that causes delay occurs between the biological signal measurement apparatus and the data analysis apparatus, the data analysis apparatus is able to obtain the trigger signal and the biological signal corresponding to the stimulation signal while having been synchronized.

A biological signal measurement system according to one embodiment of the present invention includes a biological signal measurement apparatus and a data analysis apparatus. The biological signal measurement apparatus includes a microphone that collects a sound that a subject utters and generates a sound collection signal, a sensor that measures a biological signal of a subject to the sound, and a first wireless communication portion. The data analysis apparatus includes a data processing portion that performs predetermined data processing to the biological signal, and a second wireless communication portion that performs data communication with the first wireless communication portion. The biological signal measurement apparatus includes a feature amount extraction portion that extracts a feature amount of the sound collection signal, a first trigger signal obtaining portion that obtains a first trigger signal based on the feature amount, and a synchronous sampling portion that synchronizes and samples the first trigger signal and the biological signal. The first wireless communication portion sends the first trigger signal and the biological signal synchronized and sampled with the first trigger signal to the second wireless communication portion. The data processing portion of the data analysis apparatus performs the predetermined data processing, based on the received biological signal and first trigger signal.

In this configuration, the trigger signal and the biological signal based on the sound collection signal are synchronized and sampled and are sent as a signal pair to the data analysis apparatus. Therefore, even when wireless communication that causes delay between the biological signal measurement apparatus and the data analysis apparatus, the data analysis apparatus is able to obtain the trigger (or synchronously sampled data set) signal and the biological signal corresponding to the sound collection signal while having been synchronized.

A biological signal measurement apparatus according to one embodiment of the present invention includes a trigger signal obtaining portion, a sensor, a synchronous sampling portion, and a wireless communication portion. The trigger signal obtaining portion obtains a trigger signal corresponding to a stimulation signal from outside. The sensor measures a biological signal based on reaction of a subject to the stimulation signal. The synchronous sampling portion synchronizes and samples the trigger signal and the biological signal. The wireless communication portion performs transmission and reception of data with the data analysis apparatus. The wireless communication portion receives the trigger signal from the data analysis apparatus, sends to the trigger signal obtaining portion, and sends the trigger signal and the biological signal that have been sampled by the synchronous sampling portion, to the data analysis apparatus.

In this configuration, the trigger signal and the biological signal corresponding to the stimulation signal are synchronized and sampled and are sent as a signal pair to the data analysis apparatus that performs data analysis by using these signals. Therefore, even when wireless communication that causes delay between the biological signal measurement apparatus and the data analysis apparatus, the data analysis apparatus is able to obtain the trigger signal and the biological signal corresponding to the stimulation signal while having been synchronized.

A biological signal measurement apparatus according to one embodiment of the present invention includes a microphone, a sensor, a feature amount extraction portion, a trigger signal obtaining portion, a synchronous sampling portion, and a wireless communication portion. The microphone collects a sound that a subject utters and generates a sound collection signal. The sensor measures a biological signal of a subject to the sound. The feature amount extraction portion extracts a feature amount of the sound collection signal. The trigger signal obtaining portion obtains a trigger signal based on the feature amount. The synchronous sampling portion synchronizes and samples the trigger signal and the biological signal. The wireless communication portion sends the trigger signal and the biological signal synchronized and sampled with the trigger signal, to a data analysis apparatus.

In this configuration, the trigger signal and the biological signal based on the sound collection signal are synchronized and sampled and are sent as a signal pair (or synchronously sampled data set) to the data analysis apparatus that performs data analysis by using these signals. Therefore, even when wireless communication that causes delay between the biological signal measurement apparatus and the data analysis apparatus, the data analysis apparatus is able to obtain the trigger signal and the biological signal corresponding to the sound collection signal while having been synchronized.

A data analysis apparatus according to one embodiment of the present invention includes a stimulation signal generation portion, a wireless communication portion, and a data processing portion. The stimulation signal generation portion generates a stimulation signal for giving stimulation. The wireless communication portion receives a biological signal based on reaction of a subject to the stimulation, and a trigger signal synchronously sampled with the biological signal and corresponding to the stimulation signal. The data processing portion performs predetermined data processing with respect to biological reaction corresponding to the biological signal, based on the received biological signal and trigger signal.

In this configuration, the trigger signal and biological signal corresponding to the stimulation signal synchronized and sampled are received. Therefore, data processing is able to be performed by use of the synchronized stimulation signal and biological signal.

### BRIEF DESCRIPTION OF THE DRAWINGS

FIG. 1 is a functional block diagram showing one example of a configuration of a biological signal measurement system according to a first embodiment.

FIG. 2 is a view showing a relationship on a time axis of each signal of the biological signal measurement system according to the first embodiment.

FIG. 3A and FIG. 3B are views showing one example of synchronous sampling between a trigger signal and an electroencephalogram signal.

FIG. 4 is a functional block diagram showing one example of a configuration of a biological signal measurement system according to a second embodiment.

FIG. 5 is a view showing a relationship on a time axis of each signal of the biological signal measurement system according to the second embodiment.

FIG. 6 is a functional block diagram showing one example of a configuration of a biological signal measurement system according to a third embodiment.

FIG. 7 is a view showing a relationship on a time axis of each signal of the biological signal measurement system according to the third embodiment.

FIG. 8 is a functional block diagram showing one example of a configuration of a biological signal measurement system according to a fourth embodiment.

FIG. 9 is a functional block diagram showing one example of a configuration of a biological signal measurement system according to a fifth embodiment.

FIG. 10 is a view showing a relationship on a time axis of each signal of the biological signal measurement system according to the fifth embodiment.

FIG. 11 is a functional block diagram showing one example of a configuration of a biological signal measurement system according to a sixth embodiment.

FIG. 12 is a view showing a relationship on a time axis of each signal of the biological signal measurement system according to the sixth embodiment.

FIG. 13 is a functional block diagram showing one example of a configuration of a biological signal measurement system according to a seventh embodiment.

FIG. 14 is a view showing a relationship on a time axis of each signal of the biological signal measurement system according to the seventh embodiment.

FIG. 15 is a functional block diagram showing one example of a configuration of a biological signal measurement system according to an eighth embodiment.

FIG. 16 is a view showing a relationship on a time axis of each signal of the biological signal measurement system according to the eighth embodiment.

FIG. 17 is a functional block diagram showing one example of a configuration of a biological signal measurement system according to a ninth embodiment.

FIG. 18 is a view showing a relationship on a time axis of each signal of the biological signal measurement system according to the ninth embodiment.

FIG. 19 is a functional block diagram showing one example of a configuration of a biological signal measurement system according to a tenth embodiment.

### DESCRIPTION OF EMBODIMENTS

The inventors have recognized that, in the configuration described in Patent Literature 1, it is assumed that a timing of an internal clock generated by the biological signal measurement apparatus matches a timing of an internal clock generated by the information processing apparatus. In a case in which it is difficult to reliably perform synchronization between the internal clocks of the biological signal measurement apparatus and the information processing apparatus, in order to use for the analysis of a biological signal, or the like, the synchronization between a stimulation signal and a biological signal may be unable to be reliably performed. Accordingly, the present disclosure is directed to providing a biological signal measurement system capable directed to providing a more reliably performing synchronization between a stimulation signal and a biological signal. In other words, according to the present disclosure, synchronization between a stimulation signal and a biological signal is able to be reliably performed even when communication delay occurs between a device that generates a stimulation signal and a device that obtains a biological signal.

The functionality of the elements disclosed herein may be implemented using circuitry or processing circuitry which includes general purpose processors, special purpose processors, integrated circuits, ASICs (“Application Specific Integrated Circuits”), FPGAs (“Field Programmable Gate Arrays”), conventional circuitry and/or combinations thereof which are programmed, using one or more programs stored in one or more memories, or otherwise configured to perform the disclosed functionality. Processors are considered processing circuitry or circuitry as they include transistors and other circuitry therein. The processor may be a programmed processor which executes a program which includes computer instructions stored in a memory. These computer instructions provide the logic and routines that enable the hardware (e.g., processing circuitry or circuitry) to perform the method disclosed herein. This computer program can be implemented in known formats as a computer-readable storage medium, a computer program product, a memory device, a record medium such as a CD-ROM or DVD, and/or the memory of a FPGA or ASIC.

In the disclosure, the circuitry, units, portions or means are hardware that carry out or are programmed to perform the recited functionality. The hardware may be any hardware disclosed herein which is programmed or configured to carry out the recited functionality.

### First Embodiment

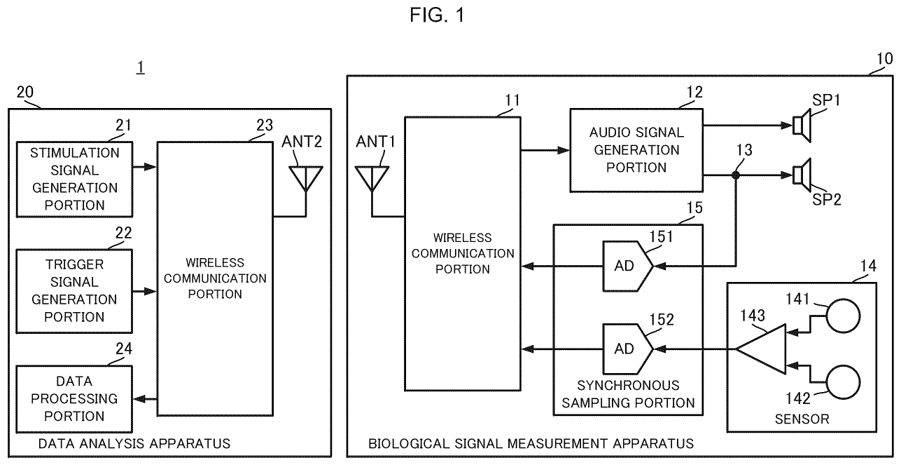

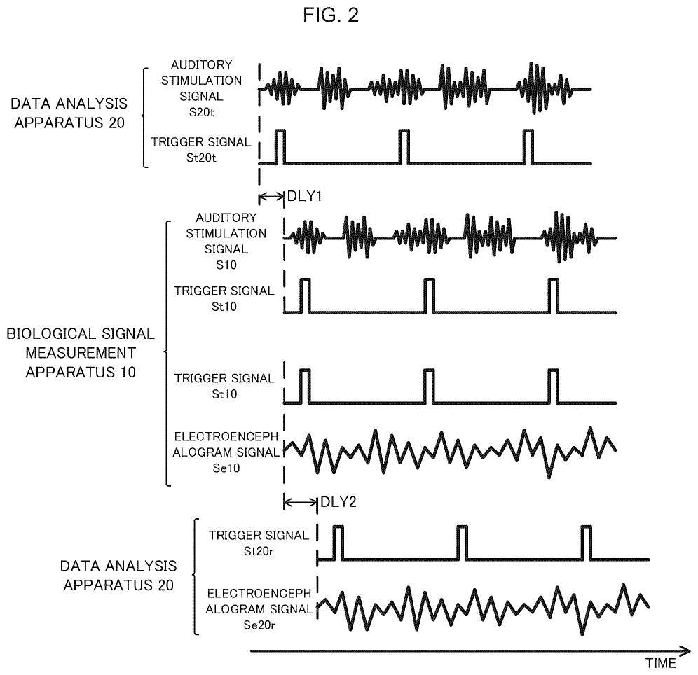

A biological signal measurement system according to a first embodiment will be described with reference to drawings. FIG. 1 is a functional block diagram showing one example of a configuration of the biological signal measurement system according to the first embodiment. FIG. 2 is a view showing a relationship on a time axis of each signal of the biological signal measurement system according to the first embodiment.

As shown in FIG. 1, the biological signal measurement system 1 includes a biological signal measurement apparatus 10 and a data analysis apparatus 20. The biological signal measurement system 1 is a system that measures an electroencephalogram signal of a subject to auditory stimulation.

### Configuration of Biological Signal Measurement Apparatus 10

The biological signal measurement apparatus 10 includes a wireless communication portion 11, an audio signal generation portion 12, a trigger signal obtaining portion 13, a sensor 14, a synchronous sampling portion 15, an antenna ANT1, a speaker SP1, and a speaker SP2. The biological signal measurement apparatus 10 is, for example, a device (a portable device) in such a size that a subject can wear. Specifically, the biological signal measurement apparatus 10 includes headphones, an earphone, a wearable device, or a processing apparatus or the like that externally connected to the sensor 14, or the like, for example. The wireless communication portion 11 corresponds to the “first wireless communication portion” of the present disclosure.

The wireless communication portion 11, the audio signal generation portion 12, and the synchronous sampling portion 15 are achieved, for example, by an electronic circuit. The speaker SP1 and the speaker SP2 are speakers of the headphones, for example, and the speaker SP1 is for an R channel and the speaker SP2 is for an L channel.

The Antenna ANT1 is connected to the wireless communication portion 11. The wireless communication portion 11 is connected to the audio signal generation portion 12 and the synchronous sampling portion 15.

The speaker SP1 and the speaker SP2 are connected to the audio signal generation portion 12. In addition, a connection line of the audio signal generation portion 12 and the speaker SP2 is branched and is connected to the synchronous sampling portion 15. A branch portion of the connection line of the audio signal generation portion 12 and the speaker SP2 corresponds to the trigger signal obtaining portion 13.

The sensor 14 is connected to the synchronous sampling portion 15.

### Configuration of Data Analysis Apparatus 20

The data analysis apparatus 20 includes a stimulation signal generation portion 21, a trigger signal generation portion 22, a wireless communication portion 23, a data processing portion 24, and an antenna ANT2. The stimulation signal generation portion 21, the trigger signal generation portion, the wireless communication portion 23, and the data processing portion 24 are achieved by an electronic circuit or an information processing apparatus such as a PC (a personal computer). Specifically, the data analysis apparatus 20 is, for example, a tablet terminal, a smartphone, or the like. The wireless communication portion 23 corresponds to the “second wireless communication portion” of the present disclosure.

The Antenna ANT2 is connected to the wireless communication portion 23. The wireless communication portion 23 is connected to the stimulation signal generation portion 21, the trigger signal generation portion 22, and the data processing portion 24.

### Specific Processing of Biological Signal Measurement System

The stimulation signal generation portion 21 generates a stimulation signal (an auditory stimulation signal S20t) for giving the stimulation (auditory stimulation) to a subject who undergoes electroencephalogram measurement. The stimulation signal generation portion 21 outputs the auditory stimulation signal S20t to the wireless communication portion 23.

The trigger signal generation portion 22 generates a trigger signal St20t of a pulse waveform repeated in a predetermined cycle. The trigger signal St20t corresponds to the “first pulse signal” of the present disclosure. The trigger signal St20t synchronizes (time synchronization) with the auditory stimulation signal S20t. The trigger signal generation portion 22 outputs the trigger signal St20t to the wireless communication portion 23.

The wireless communication portion 23 converts the auditory stimulation signal S20t and the trigger signal St20t into a wireless communication data format, and sends from the antenna ANT2. The wireless communication portion 11 receives the auditory stimulation signal S20t and the trigger signal St20t that have been received by the antenna ANT1. In other words, the wireless communication portion 23 and the wireless communication portion 11 are able to perform data communication and the auditory stimulation signal S20t and the trigger signal St20t are transmitted and received between the wireless communication portion 23 and the wireless communication portion 11.

The wireless communication portion 11 converts the auditory stimulation signal S20t and the trigger signal St20t into a data format for sound emission and outputs the data format to the audio signal generation portion 12.

The audio signal generation portion 12 demodulates the auditory stimulation signal S20t and generates an auditory stimulation signal S10 of an analog signal. The audio signal generation portion 12 demodulates the trigger signal St20t and generates a trigger signal St10 of an analog signal.

The audio signal generation portion 12 outputs the auditory stimulation signal S10 of the analog signal to the speaker SP1. The trigger signal St10 of the analog signal is output to the speaker SP2. On this occasion, the audio signal generation portion 12 synchronizes and outputs the auditory stimulation signal S10 and the trigger signal St10.

The trigger signal St10 output from the audio signal generation portion 12 is distributed by the trigger signal obtaining portion 13 and is input into the synchronous sampling portion 15. In other words, the trigger signal obtaining portion 13 has a function to obtain the trigger signal St10 output from the audio signal generation portion 12 and to make output to the synchronous sampling portion 15.

The sensor 14 includes an electrode 141, an electrode 142, and an amplifier 143. The electrode 141 and the electrode 142 are worn on the head (a place in which the electroencephalogram measurement is performed) of a subject.

The electrode 141 and the electrode 142 detect an electroencephalogram signal Se10, and make output to the amplifier 143. The amplifier 143 amplifies the electroencephalogram signal Se10 and makes output to the synchronous sampling portion 15.

It is to be noted that the electroencephalogram signal Se10 (the biological signal) measured by the sensor 14 is a signal based on reaction of the subject to auditory stimulation (stimulation) of a sound emitted from the speaker SP1. In other words, the electroencephalogram signal Se10 detected by the sensor 14 is continuously measured over time and is a signal including a waveform corresponding to this, when the subject reacts to the auditory stimulation.

The synchronous sampling portion 15 includes a sampling device 151 and a sampling device 152. The sampling device 151 and the sampling device 152 perform AD sampling. The AD sampling is to sample an analog signal as a digital signal.

The sampling device 151 performs digital sampling on the trigger signal St10. The sampling device 152 performs the digital sampling on the electroencephalogram signal Se10.

The sampling device 151 and the sampling device 152 synchronize and perform sampling. In other words, the trigger signal St10 to be sampled by the sampling device 151 and the electroencephalogram signal Se10 to be sampled by the sampling device 152 are synchronized.

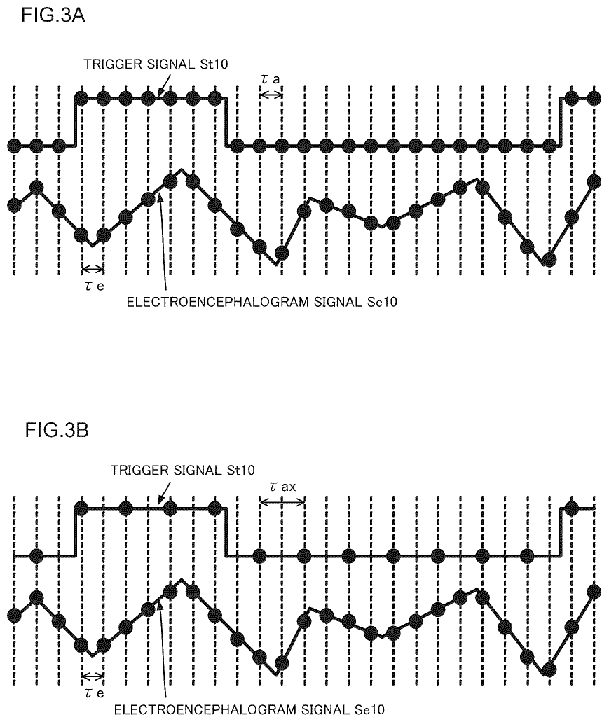

FIG. 3A and FIG. 3B are views showing one example of synchronous sampling between the trigger signal and the electroencephalogram signal.

In FIG. 3A, a sampling cycle τa of the trigger signal St10 is the same as a sampling cycle τe of the electroencephalogram signal Se10. In a case shown in FIG. 3A, a sampling timing of the trigger signal St10 matches a sampling timing of the electroencephalogram signal Se10.

In FIG. 3B, a sampling cycle τax of the trigger signal St10 is twice the sampling cycle τe of the electroencephalogram signal Se10. In a case shown in FIG. 3B, the sampling timing of the trigger signal St10 matches one out of two times of the sampling timing of the electroencephalogram signal Se10. In this manner, the synchronous sampling is the timing at which the trigger signal St10 is sampled, and means sampling the electroencephalogram signal Se10. It is to be noted that the sampling cycle of the trigger signal St10, although being also possible to increase three or more times of the sampling cycle τe of the electroencephalogram signal Se10, may be either one or two times since a synchronization error is increased as multiples are increased.

The synchronous sampling portion 15 outputs the synchronously sampled trigger signal St10 and electroencephalogram signal Se10 (collectively referred to herein as a synchronously sampled data set), to the wireless communication portion 11.

The wireless communication portion 11 converts the synchronously sampled trigger signal St10 and electroencephalogram signal Se10 into a data format for wireless communication and sends from the antenna ANT1. The wireless communication portion 23 receives the trigger signal St10 and the electroencephalogram signal Se10 that have been received by the antenna ANT2. In other words, the trigger signal St10 and the electroencephalogram signal Se10 are transmitted and received between the wireless communication portion 11 and the wireless communication portion 23.

The wireless communication portion 23 converts the trigger signal St10 and the electroencephalogram signal Se10 into a data format for arithmetic processing and generates a trigger signal St20r and an electroencephalogram signal Se20r. The wireless communication portion 23 outputs the trigger signal St20r and the electroencephalogram signal Se20r to the data processing portion 24.

The data processing portion 24 performs predetermined data processing, based on the trigger signal St20r and the electroencephalogram signal Se20r. The predetermined data processing is, for example, processing to detect an event-related potential of P300 or the like. It is to be noted that the predetermined data processing may be other processing as long as the data processing is to analyze brain waves to be generated to auditory stimulation.

In such a configuration, even when the data communication between the data analysis apparatus 20 and the biological signal measurement apparatus 10 is performed by wireless communication with a delay, the data processing portion 24 is able to obtain the trigger signal and the electroencephalogram signal based on the auditory stimulation in a time-synchronized state. Herein, the data processing portion 24 grasps a time relationship between the auditory stimulation signal and the trigger signal. In other words, the data processing portion 24 grasps the auditory stimulation signal and the trigger signal in a synchronized state.

Accordingly, the data processing portion 24 is able to achieve time synchronization between the auditory stimulation signal and the electroencephalogram signal.

Specifically, for example, as shown in FIG. 2, data transmission from the data analysis apparatus 20 to the biological signal measurement apparatus 10 has a delay time DLY1. Further, the data transmission from the biological signal measurement apparatus 10 to the data analysis apparatus 20 has a delay time DLY2. Accordingly, time from when the data analysis apparatus 20 outputs the auditory stimulation signal until when obtaining the electroencephalogram signal Se20r has a delay time (DLY1+DLY2).

Therefore, when the synchronous sampling as in the present embodiment is not performed, a time difference (the delay time (DLY1+DLY2)) occurs between output timing of the auditory stimulation signal in the data analysis apparatus 20 and obtaining timing of the electroencephalogram signal Se20r.

However, the biological signal measurement system 1 according to the present embodiment achieves the synchronous sampling by the biological signal measurement apparatus 10, transmission and reception of the trigger signal and the electroencephalogram signal in the synchronized state between the biological signal measurement apparatus 10 and the data analysis apparatus 20, and grasping of time-synchronization between the auditory stimulation signal and the electroencephalogram signal in the data analysis apparatus 20.

Accordingly, the data processing portion 24, when a delay time due to a communication failure or processing time occurs at the time of data processing, is able to achieve time-synchronization by synchronizing each of the auditory stimulation signal and the electroencephalogram signal with the trigger signal. According to this, the data processing portion 24 is able to correctly perform analysis of brain waves to be generated to the auditory stimulation. In particular, it is effective when dealing with a biological signal to be generated in constant time to stimulation, as in the event-related potential.

It is to be noted that each embodiment including the present embodiment describes the electroencephalogram signal as an example, as a biological signal based on reaction of a subject to stimulation. However, as the biological signal, a signal based on a pulse, an electrocardiographic signal, a myoelectric signal, or the like is also able to be employed corresponding to a contact position of the sensor 14 and skin. In this case, the data processing portion 24 performs data processing to analyze the biological reaction corresponding to a type of each signal. The sampling rate of the synchronous sampling circuitry 15 is adjustable depending on the type of biological signal being measured (e.g., lower for heart rate, higher for EEG).

### Second Embodiment

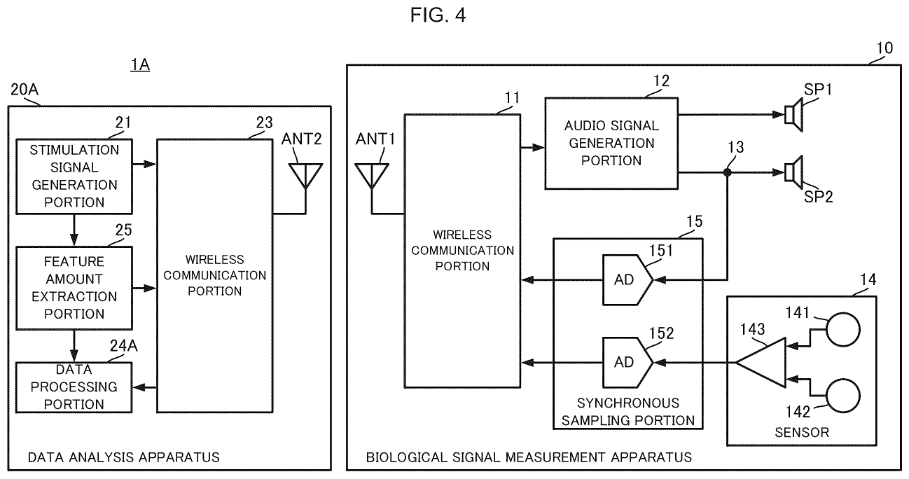

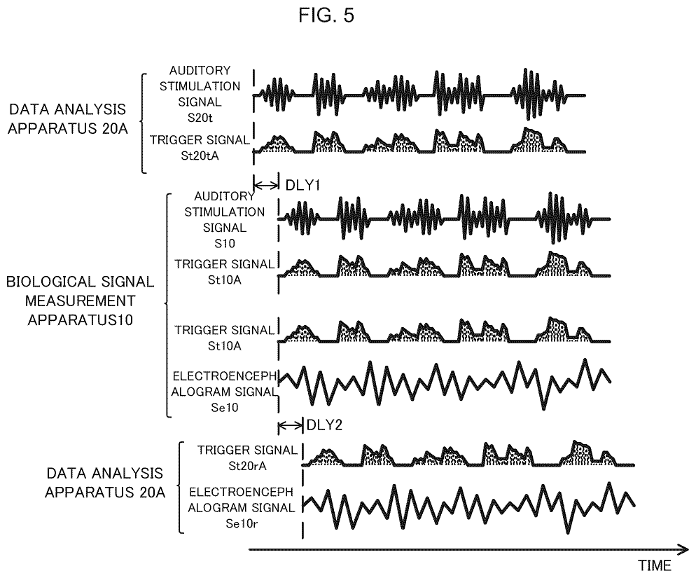

A biological signal measurement system according to a second embodiment will be described with reference to drawings. FIG. 4 is a functional block diagram showing one example of a configuration of the biological signal measurement system according to the second embodiment. FIG. 5 is a view showing a relationship on a time axis of each signal of the biological signal measurement system according to the second embodiment.

As shown in FIG. 4 and FIG. 5, the biological signal measurement system 1A according to the second embodiment is different from the biological signal measurement system 1 according to the first embodiment in that a data analysis apparatus 20A is provided and in that a feature amount of an auditory stimulation signal is used as a trigger signal.

Other configurations of the biological signal measurement system 1A are the same as or similar to the configurations of the biological signal measurement system 1, and a description of the same or similar configurations will be omitted.

The biological signal measurement system 1A includes a data analysis apparatus 20A. The data analysis apparatus 20A is different from the data analysis apparatus 20 in that a data processing portion 24A and a feature amount extraction portion 25 are provided.

The feature amount extraction portion 25 (a first feature amount extraction portion) extracts the feature amount, e.g., a signal characteristic such as an amplitude envelope, of the auditory stimulation signal S20t to be output from the stimulation signal generation portion 21. In the present disclosure, the term “feature amount” (or “feature value”) refers to any characteristic, parameter, or statistical measure derived from a signal (e.g., the stimulation signal or sound collection signal) that represents a property of the signal. The feature amount is not limited to an amplitude envelope, but may include, for example, a signal magnitude (e.g., peak amplitude, RMS power), a frequency domain characteristic (e.g., a specific frequency component, spectral centroid, or band power), a temporal characteristic (e.g., zero-crossing rate, rise time, or pulse width), a phase characteristic, or a complex feature vector derived from processing such as Fast Fourier Transform (FFT) or wavelet transform. Furthermore, the feature amount extraction circuitry may be configured to generate the feature amount by detecting a specific pattern, watermark, or pilot signal embedded within the stimulation signal. The feature amount extraction portion 25, for example, extracts an envelope of the auditory stimulation signal S20t and generates a trigger signal St20tA. The feature amount extraction portion 25, for example, is configured by full wave rectification and a low-pass filter. The feature amount extraction portion 25 outputs the trigger signal St20tA made of the feature amount, to the wireless communication portion 23. The trigger signal St20tA made of the feature amount is data set arranged in time series.

The wireless communication portion 23 sends the auditory stimulation signal S20t and the trigger signal St20tA made of the feature amount in the synchronized state, to the wireless communication portion 11 of the biological signal measurement apparatus 10.

The wireless communication portion 11 receives the auditory stimulation signal S20t and the trigger signal St20tA made of the feature amount. The synchronous sampling portion 15 performs the synchronous sampling between a trigger signal St10A demodulated from the trigger signal St20tA, and the electroencephalogram signal Se10. The wireless communication portion 11 sends the synchronously sampled trigger signal St10A and electroencephalogram signal Se10, to the wireless communication portion 23.

The wireless communication portion 23 receives the trigger signal St10A and the electroencephalogram signal Se10 and demodulates to a trigger signal St20rA and the electroencephalogram signal Se20r. The data processing portion 24A performs the predetermined data processing, based on the trigger signal St20rA and the electroencephalogram signal Se20r.

In this manner, the biological signal measurement system 1A is able to produce the same or similar operational effect as the biological signal measurement system 1. In addition, the biological signal measurement system 1A, since using the feature amount generated from the auditory stimulation signal, does not need to separately generate the trigger signal of a pulse signal.

In addition, the following operational effect is able to be obtained by use of the feature amount. The frequency band of the auditory stimulation signal is higher than the frequency band of the electroencephalogram signal. Therefore, when the sampling rate of the electroencephalogram signal is matched with the sampling rate of the auditory stimulation signal, the sampling rate of the electroencephalogram signal becomes higher than necessary to the analysis of brain waves, which increases data traffic. On the other hand, the feature amount, for example, when being an envelope of the auditory stimulation signal, serves as a frequency band lower than the auditory stimulation signal.

Therefore, by using the feature amount, the biological signal measurement system 1 enables the analysis of brain waves and is able to keep the sampling rate of the electroencephalogram signal low. Accordingly, the biological signal measurement system 1 is able to significantly reduce the data traffic between the biological signal measurement apparatus 10 and the data analysis apparatus 20A. In particular, this effect is effective at the time of data transmission from the biological signal measurement apparatus 10 to the data analysis apparatus 20A.

It is to be noted that the present embodiment shows an aspect in which the envelope of the auditory stimulation signal is used as the feature amount. However, the feature amount is not limited to the envelope as long as being able to be obtained based on the auditory stimulation signal.

In a case in which extraction of the feature amount does not take time, that is, in a case in which the trigger signal is not delayed from the auditory stimulation signal by the extraction of the feature amount (in a case of a time error range unaffected by the analysis of brain waves), the feature amount extraction portion 25 may extract the feature amount from the auditory stimulation signal S20t to be output from the stimulation signal generation portion 21 and may generate the trigger signal St20tA.

On the other hand, in a case in which the extraction of the feature amount takes time, that is, in a case in which the trigger signal is largely delayed from the auditory stimulation signal by the extraction of the feature amount (in a case of a delay affected by the analysis of brain waves), the feature amount extraction portion 25 extracts the feature amount from the auditory stimulation signal S20t in advance and stores as the trigger signal St20tA. Then, the feature amount extraction portion 25 may output the trigger signal St20tA in synchronization with output timing of the auditory stimulation signal S20t from the stimulation signal generation portion 21.

### Third Embodiment

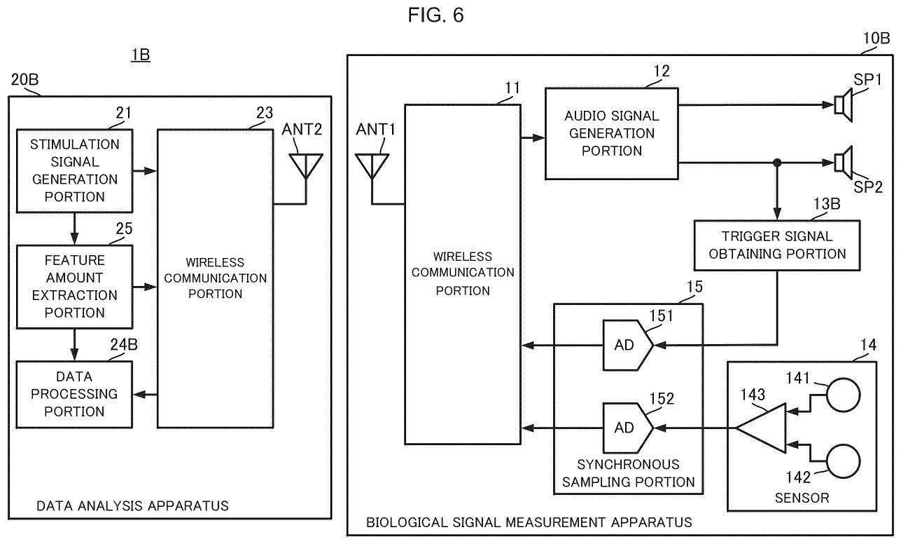

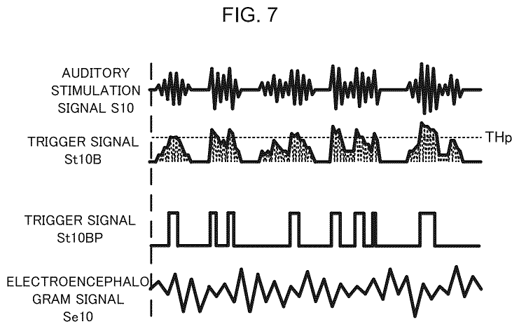

A biological signal measurement system according to a third embodiment will be described with reference to drawings. FIG. 6 is a functional block diagram showing one example of a configuration of the biological signal measurement system according to the third embodiment. FIG. 7 is a view showing a relationship on a time axis of each signal of the biological signal measurement system according to the third embodiment.

As shown in FIG. 6 and FIG. 7, the biological signal measurement system 1B according to the third embodiment is different from the biological signal measurement system 1A according to the second embodiment in that a biological signal measurement apparatus 10B and a data analysis apparatus 20B are provided and in that a pulse signal based on the feature amount of an auditory stimulation signal is used as a trigger signal. Other configurations of the biological signal measurement system 1B are the same as or similar to the configurations of the biological signal measurement system 1A, and a description of the same or similar configurations will be omitted.

The biological signal measurement system 1B includes a biological signal measurement apparatus 10B and a data analysis apparatus 20B.

The biological signal measurement apparatus 10B is different in that a trigger signal obtaining portion 13B is provided. The data analysis apparatus 20B is different from the data analysis apparatus 20 in that a data processing portion 24B is provided.

The trigger signal obtaining portion 13B generates a second pulse signal of a pulse waveform binarized by whether or not a predetermined condition is satisfied from the feature amount, and obtains the second pulse signal as a trigger signal St10B.

More specifically, the trigger signal obtaining portion 13B stores a threshold value THp for generation of the second pulse signal. The trigger signal obtaining portion 13B compares the level of the trigger signal St10B of the feature amount to be output from the audio signal generation portion 12 to the speaker SP2, and the threshold value THp.

The trigger signal obtaining portion 13B defines a period equal to or greater than the threshold value THp in the trigger signal St10B as a Hi level and defines a period less than the threshold value THp in the trigger signal St10B as a Low level, and then generates and obtains the second pulse signal as a trigger signal St10BP.

The trigger signal obtaining portion 13B outputs the trigger signal St10BP of the second pulse signal to the synchronous sampling portion 15.

The synchronous sampling portion 15 performs synchronous sampling between the trigger signal St10BP of the second pulse signal and the electroencephalogram signal Se10, and makes output to the wireless communication portion 11. The wireless communication portion 11 sends the synchronously sampled trigger signal St10BP and electroencephalogram signal Se10, to the wireless communication portion 23.

The wireless communication portion 23 receives the trigger signal St10BP of the second pulse signal, and the electroencephalogram signal Se10 and makes output to the data processing portion 24B.

The trigger signal St10B of the feature amount is inputted from the feature amount extraction portion 25 into the data processing portion 24B. The data processing portion 24B stores the same or similar threshold value THp as in the trigger signal obtaining portion 13B. The data processing portion 24B compares the trigger signal St10B and the threshold value THp and generates a second pulse signal for synchronization during analysis.

The data processing portion 24B, by synchronizing the second pulse signal for synchronization during analysis and the trigger signal of the second pulse signal received from the biological signal measurement apparatus 10B so as to be matched with each other, synchronizes the electroencephalogram signal and the stimulation signal, and then performs the predetermined data processing.

In this manner, the biological signal measurement system 1B is able to produce the same or similar operational effect as the biological signal measurement system 1A. In addition, a binarized trigger signal is sent from biological signal measurement apparatus 10B to the data analysis apparatus 20B. Accordingly, the data traffic at the time of transmission from the biological signal measurement apparatus 10B to the data analysis apparatus 20B is able to be reduced.

### Fourth Embodiment

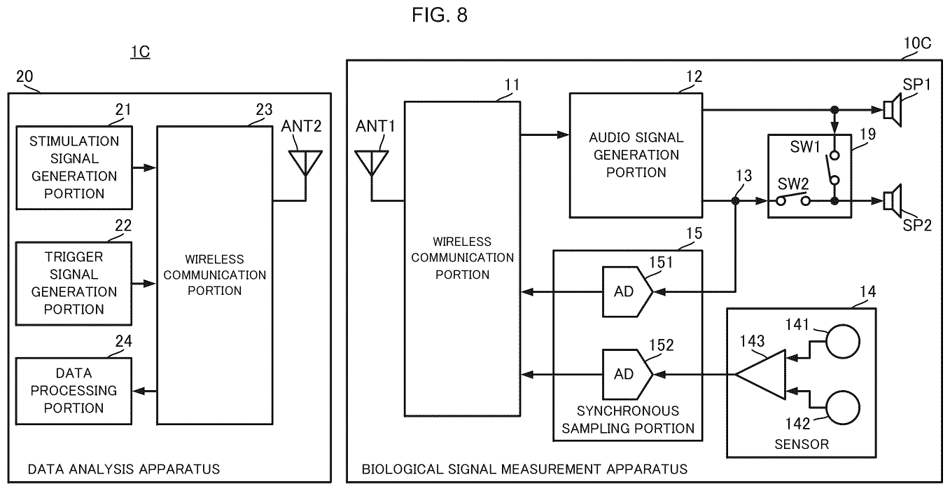

A biological signal measurement system according to a fourth embodiment will be described with reference to drawings. FIG. 8 is a functional block diagram showing one example of a configuration of the biological signal measurement system according to the fourth embodiment.

As shown in FIG. 8, the biological signal measurement system 1C according to the fourth embodiment is different from the biological signal measurement system 1 according to the first embodiment in that a biological signal measurement apparatus 10C is provided. Other configurations of the biological signal measurement system 1C are the same as or similar to the configurations of the biological signal measurement system 1, and a description of the same or similar configurations will be omitted. The biological signal measurement apparatus 10C is different from the biological signal measurement apparatus 10 according to the first embodiment in that a switch circuit 19 is provided. Other configurations of the biological signal measurement apparatus 10C are the same as or similar to the configurations of the biological signal measurement apparatus 10, and a description of the same or similar configurations will be omitted.

The switch circuit 19 includes a switch element SW1 and a switch element SW2. The switch element SW1 is connected between a first connection line that connects the audio signal generation portion 12 and the speaker SP1 (a first output portion or interface), and a second connection line that connects the audio signal generation portion 12 and the speaker SP2 (a second output portion or interface). The switch element SW2 is connected between the trigger signal obtaining portion 13 in the second connection line, and a node to the switch element SW1.

The switch circuit 19 switches a first connection mode and a second connection mode. The switch circuit 19 executes the first connection mode during a period when the electroencephalogram measurement is performed, and executes the second connection mode during a period when no electroencephalogram measurement is performed. In other words, the first Connection Mode as a measurement mode (where stimulation goes to one ear and trigger to the system) and the second connection mode is an entertainment mode (where distinct audio goes to both ears).

In the first connection mode, the switch circuit 19 short-circuits the switch element SW1 and opens the switch element SW2. In this case, the switch circuit 19 transmits the auditory stimulation signal from the audio signal generation portion 12 to the speaker SP1 and the speaker SP2. In addition, the switch circuit 19 outputs the trigger signal from the audio signal generation portion 12 only to the synchronous sampling portion 15.

According to this, auditory stimulation is able to be given even to a person who has hearing impairment in one ear. In addition, the trigger signal is able to be prevented from being output to the speaker SP2 as an unnecessary noise.

In the second connection mode, the switch circuit 19 opens the switch element SW1 and short-circuits the switch element SW2. In this case, the switch circuit 19 transmits a different auditory stimulation signal from the audio signal generation portion 12 to the speaker SP1 and the speaker SP2. According to this, for example, during music reproduction, two types of auditory stimulation signals are able to be individually output to the speaker SP1 and the speaker SP2, so that stereophonic reproduction is achieved as in the ordinary headphones.

### Fifth Embodiment

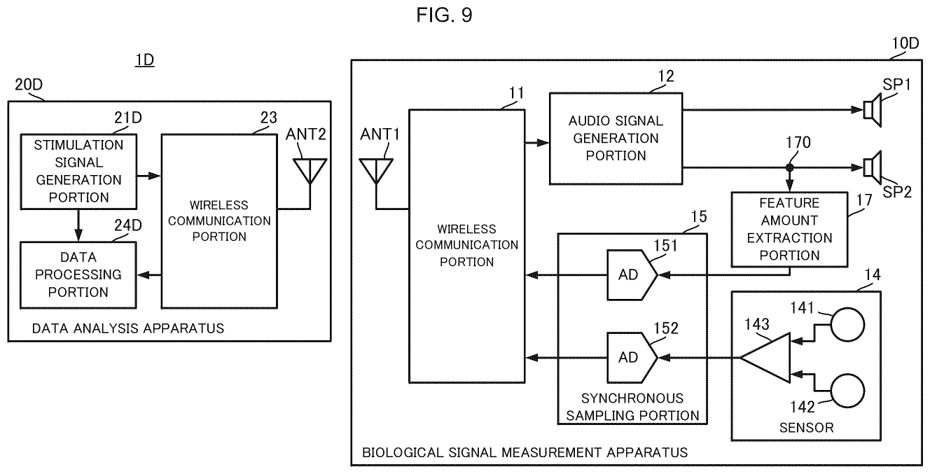

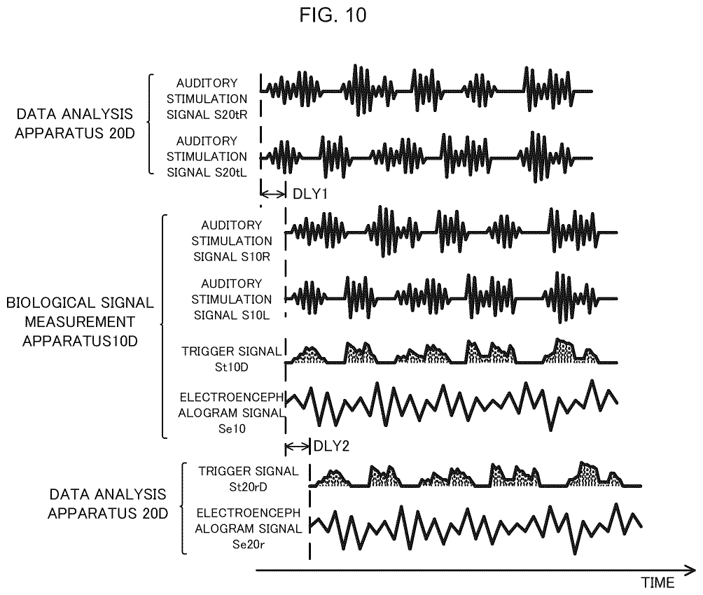

A biological signal measurement system according to a fifth embodiment will be described with reference to drawings. FIG. 9 is a functional block diagram showing one example of a configuration of the biological signal measurement system according to the fifth embodiment. FIG. 10 is a view showing a relationship on a time axis of each signal of the biological signal measurement system according to the fifth embodiment.

As shown in FIG. 9 and FIG. 10, the biological signal measurement system 1D according to the fifth embodiment is different from the biological signal measurement system 1A according to the second embodiment in that a biological signal measurement apparatus 10D and a data analysis apparatus 20D are provided and in that a feature amount of an auditory stimulation signal is extracted by the biological signal measurement apparatus 10D. Other configurations of the biological signal measurement system 1D are the same as or similar to the configurations of the biological signal measurement system 1A, and a description of the same or similar configurations will be omitted.

The biological signal measurement system 1D includes a biological signal measurement apparatus 10D and a data analysis apparatus 20D.

The biological signal measurement apparatus 10D is different from the biological signal measurement apparatus 10 in that a feature amount extraction portion 17 and a distribution circuit 170 are provided. The data analysis apparatus 20D is different from the data analysis apparatus 20A in that a stimulation signal generation portion 21D and a data processing portion 24D are provided.

The stimulation signal generation portion 21D generates auditory stimulation signals S20tR and S20tL of two channels. The wireless communication portion 23 sends the auditory stimulation signals S20tR and S20tL of two channels.

The wireless communication portion 11 receives the auditory stimulation signals S20tR and S20tL of two channels, performs data format conversion and generates auditory stimulation signal S10R and S10L, and makes output to the audio signal generation portion 12. The audio signal generation portion 12 outputs the auditory stimulation signal S10R to the speaker SP1 and outputs the auditory stimulation signal S10L to the speaker SP2.

The feature amount extraction portion 17 (a second feature amount extraction portion) obtains the auditory stimulation signal S10L through the distribution circuit 170. The feature amount extraction portion 17 extracts a feature amount of the auditory stimulation signal S10L and generates a trigger signal St10D. The feature amount extraction portion 17 outputs the trigger signal St10D to the synchronous sampling portion 15. The distribution circuit 170 also serves to obtain a signal as a trigger and is one example of the trigger signal obtaining portion.

The synchronous sampling portion 15 performs synchronous sampling between the trigger signal St10D of the feature amount and the electroencephalogram signal Se10, and makes output to the wireless communication portion 11. The wireless communication portion 11 sends the synchronously sampled trigger signal St10D of the feature amount and electroencephalogram signal Se10, to the wireless communication portion 23.

The wireless communication portion 23 performs the data format conversion of the synchronously sampled trigger signal St10D of the feature amount and electroencephalogram signal Se10 and generates a trigger signal St20rD and the electroencephalogram signal Se20r, and makes output to the data processing portion 24D.

The auditory stimulation signal S20tL is input into the data processing portion 24D. The data processing portion 24D extracts the feature amount of the auditory stimulation signal S20tL and generates a signal equivalent to the trigger signal St20rD.

The data processing portion 24D performs the predetermined data processing based on the trigger signal St20rD and the electroencephalogram signal Se20r that have been received from the biological signal measurement apparatus 10D and the signal equivalent to the trigger signal St20rD generated by the data processing portion 24D.

In this manner, the biological signal measurement system 1D is able to produce the same or similar operational effect as the biological signal measurement system 1. In addition, the biological signal measurement system 1D, in the biological signal measurement apparatus 10D, extracts a feature amount in a system other than a sound emission system and generates a trigger signal. According to this, the data analysis apparatus 20D does not need to generate a trigger signal and send to the biological signal measurement apparatus 10D.

In this case, for example, a stereo sound signal of two channels is able to be used as the auditory stimulation signal. Accordingly, the biological signal measurement apparatus 10D is able to be used as a music player of stereophonic reproduction while achieving the measurement of the electroencephalogram signal.

### Sixth Embodiment

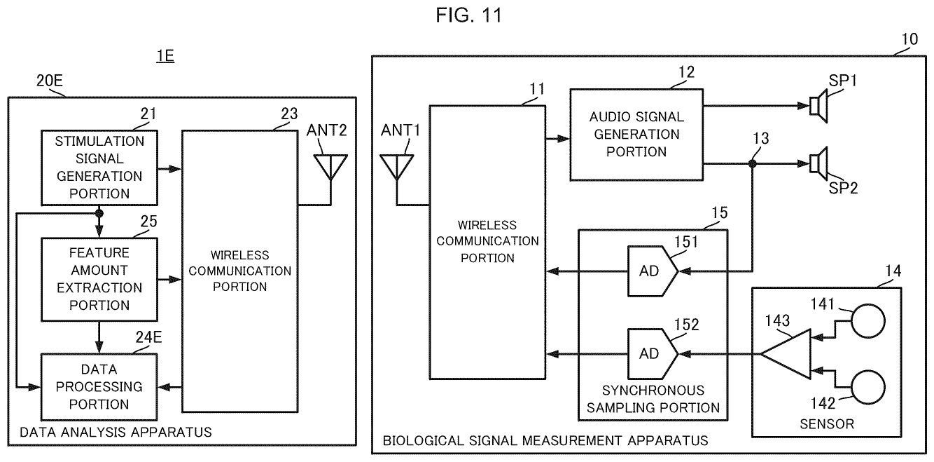

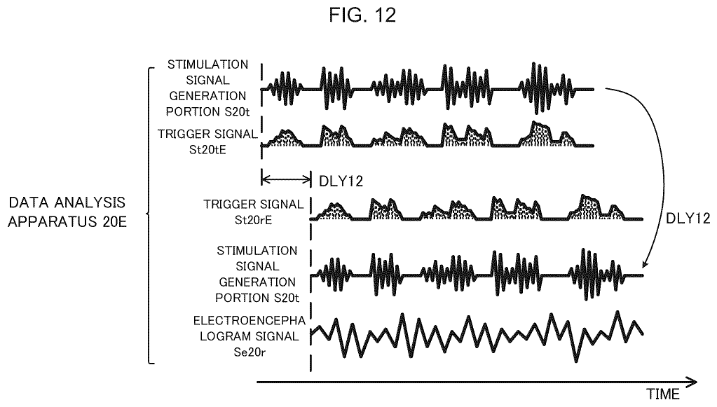

A biological signal measurement system according to a sixth embodiment will be described with reference to drawings. FIG. 11 is a functional block diagram showing one example of a configuration of the biological signal measurement system according to the sixth embodiment. FIG. 12 is a view showing a relationship on a time axis of each signal of the biological signal measurement system according to the sixth embodiment.

As shown in FIG. 11 and FIG. 12, the biological signal measurement system 1E according to the sixth embodiment is different from the biological signal measurement system 1A according to the second embodiment in that a data analysis apparatus 20E is provided and in that a time relationship among an auditory stimulation signal, a feature amount (a trigger signal), and an electroencephalogram signal is able to be estimated. Other configurations of the biological signal measurement system 1E are the same as or similar to the configurations of the biological signal measurement system 1A, and a description of the same or similar configurations will be omitted.

The biological signal measurement system 1E includes the biological signal measurement apparatus 10 and a data analysis apparatus 20E. The data analysis apparatus 20E is different from the data analysis apparatus 20A in that a data processing portion 24E is provided.

A trigger signal St20rE of the feature amount and the electroencephalogram signal Se20r that have been received from the biological signal measurement apparatus 10 are input into the data processing portion 24E, through the wireless communication portion 23. In addition, the auditory stimulation signal S20t is input from stimulation signal generation portion 21 into the data processing portion 24E.

The feature amount extraction portion 25 extracts the feature amount of the auditory stimulation signal S20tL input from the stimulation signal generation portion 21 and generates the trigger signal St20tE. The trigger signal St20tE of the feature amount generated by the feature amount extraction portion 25 is input into the data processing portion 24E.

The data processing portion 24E includes a delay time estimation portion. The delay time estimation portion estimates delay time DLY12 of the trigger signal St20rE (the trigger signal received from the biological signal measurement apparatus 10) of the feature amount to the auditory stimulation signal S20t.

The data processing portion 24E performs time synchronization between the auditory stimulation signal S20t and the electroencephalogram signal Se20r, based on the estimated delay time DLY12.

More specifically, the auditory stimulation signal S20t and the trigger signal St20tE that have been generated by the data analysis apparatus 20E are synchronized with each other. The trigger signal St20rE from the biological signal measurement apparatus 10E is a signal based on the trigger signal St20tE of the data analysis apparatus 20E. Accordingly, the data processing portion 24E compares waveforms of the trigger signal St20rE and the trigger signal St20tE and is able to obtain the delay time DLY12 of the trigger signal St20rE to the trigger signal St20tE. More specifically, the time difference that causes the mutual correlation coefficients of the trigger signal St20tE and the trigger signal St20rE to become the largest is considered to have been synchronized. In other words, the data processing circuitry calculates a cross-correlation between the transmitted trigger signal and the received trigger signal to determine the time lag (latency) where the correlation is maximized.

Then, based on the trigger signal St20tE and the auditory stimulation signal S20t synchronizing, the data processing portion 24E is able to estimate the delay time DLY12 of the trigger signal St20rE to the auditory stimulation signal S20t.

Furthermore, since the trigger signal St20rE and the electroencephalogram signal Se20r synchronize with each other, the data processing portion 24E is able to synchronize the auditory stimulation signal S20t and the electroencephalogram signal Se20r, based on the delay time DLY12.

According to such a configuration, the data processing portion 24E is able to perform the predetermined data processing in a state in which the auditory stimulation signal S20t and the electroencephalogram signal Se20r are synchronized with each other. According to this, the biological signal measurement system 1E is able to more directly measure the electroencephalogram signal to stimulation.

### Seventh Embodiment

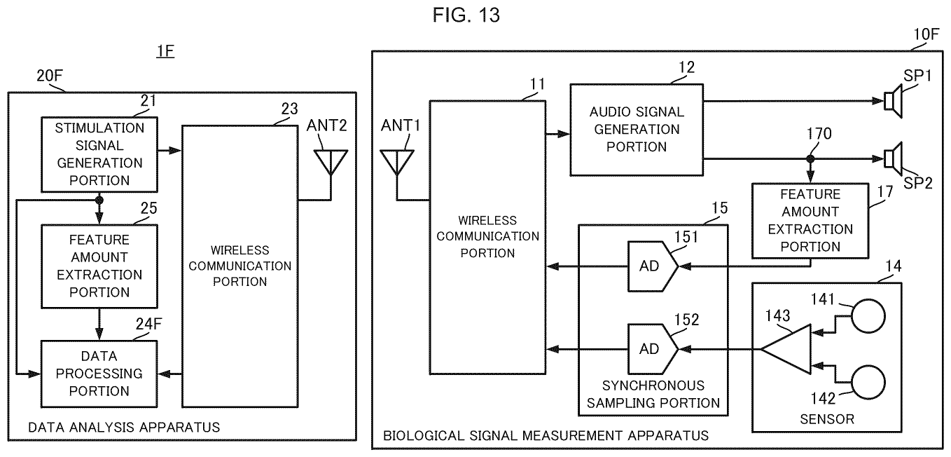

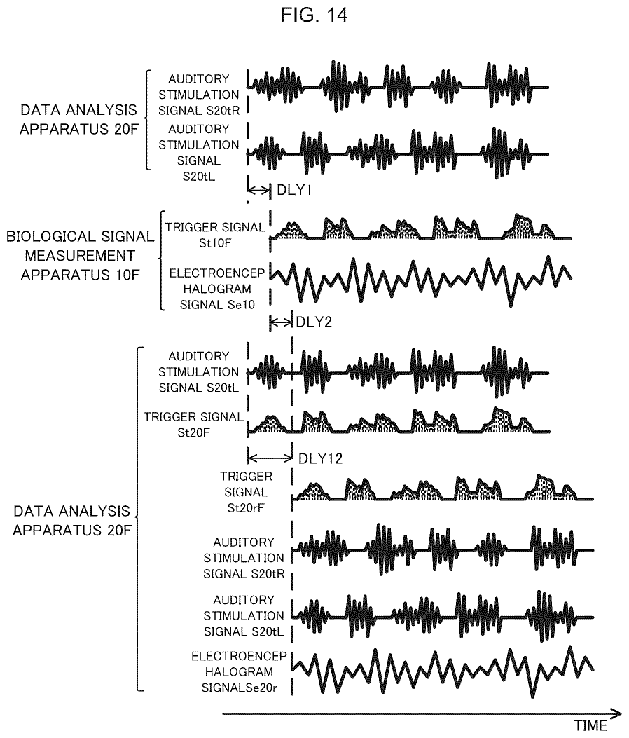

A biological signal measurement system according to a seventh embodiment will be described with reference to drawings. FIG. 13 is a functional block diagram showing one example of a configuration of the biological signal measurement system according to the seventh embodiment. FIG. 14 is a view showing a relationship on a time axis of each signal of the biological signal measurement system according to the seventh embodiment.

As shown in FIG. 13 and FIG. 14, the biological signal measurement system 1F according to the seventh embodiment is different from the biological signal measurement system 1D according to the fifth embodiment in that a data analysis apparatus 20F is provided and in that a time relationship among an auditory stimulation signal, a feature amount (a trigger signal), and an electroencephalogram signal is able to be estimated. Other configurations of the biological signal measurement system 1F are the same as or similar to the configurations of the biological signal measurement system 1D, and a description of the same or similar configurations will be omitted.

The biological signal measurement system 1F includes a biological signal measurement apparatus 10F and a data analysis apparatus 20F. The biological signal measurement apparatus 10F includes substantially the same configurations as the biological signal measurement apparatus 10D.

The data analysis apparatus 20F is different from the data analysis apparatus 20D in that a data processing portion 24F and the feature amount extraction portion 25 are provided.

The feature amount extraction portion 25 extracts the feature amount of the auditory stimulation signal S20tL generated by the stimulation signal generation portion 21 and generates a trigger signal St20F. The feature amount extraction portion 25 outputs the trigger signal St20F to the data processing portion 24F.

A trigger signal St20rF of the feature amount and the electroencephalogram signal Se20r that have been received from the biological signal measurement apparatus 10 are input into the data processing portion 24F, through the wireless communication portion 23. In addition, the auditory stimulation signal S20tL is input from the stimulation signal generation portion 21, and the trigger signal St20F of the feature amount is input from the feature amount extraction portion 25, into the data processing portion 24F.

The data processing portion 24F includes the delay time estimation portion. The delay time estimation portion estimates delay time DLY12 of the trigger signal St20rF (the trigger signal received from the biological signal measurement apparatus 10F) of the feature amount to the auditory stimulation signal S20tL.

The data processing portion 24F performs time synchronization between the auditory stimulation signal S20tL and the electroencephalogram signal Se20r, based on the estimated delay time DLY12.

More specifically, the auditory stimulation signal S20tL and the trigger signal St20F that have been generated by the data analysis apparatus 20F are synchronized with each other. The trigger signal St20rF from the biological signal measurement apparatus 10F is based on the auditory stimulation signal S20tL of the data analysis apparatus 20F and is a signal based on the same original signal as the trigger signal St20F. Accordingly, the data processing portion 24F compares waveforms of the trigger signal St20rF and the trigger signal St20F and is able to obtain the delay time DLY12 of the trigger signal St20rF to the trigger signal St20F.

Then, based on the trigger signal St20F and the auditory stimulation signal S20tL synchronizing, the data processing portion 24F is able to estimate the delay time DLY12 of the trigger signal St20rF to the auditory stimulation signal S20tL.

Further, since the trigger signal St20rF and the electroencephalogram signal Se20r synchronize with each other, the data processing portion 24F is able to synchronize the auditory stimulation signal S20tL and the electroencephalogram signal Se20r, based on the delay time DLY12.

According to such a configuration, the data processing portion 24F is able to perform the predetermined data processing in a state in which the auditory stimulation signal S20tL and the electroencephalogram signal Se20r are synchronized with each other. According to this, the biological signal measurement system 1F is able to more directly measure the electroencephalogram signal to stimulation.

Furthermore, the auditory stimulation signal S20tL and the auditory stimulation signal S20tR are a pair of signals for performing stereophonic reproduction, for example, and are synchronized with each other. Accordingly, the data processing portion 24F is able to perform the predetermined data processing in a state in which the auditory stimulation signal S20tR and the electroencephalogram signal Se20r are synchronized with each other. According to this, for example, the biological signal measurement system 1F is able to more directly measure the electroencephalogram signal to a stereo sound.

### Eighth Embodiment

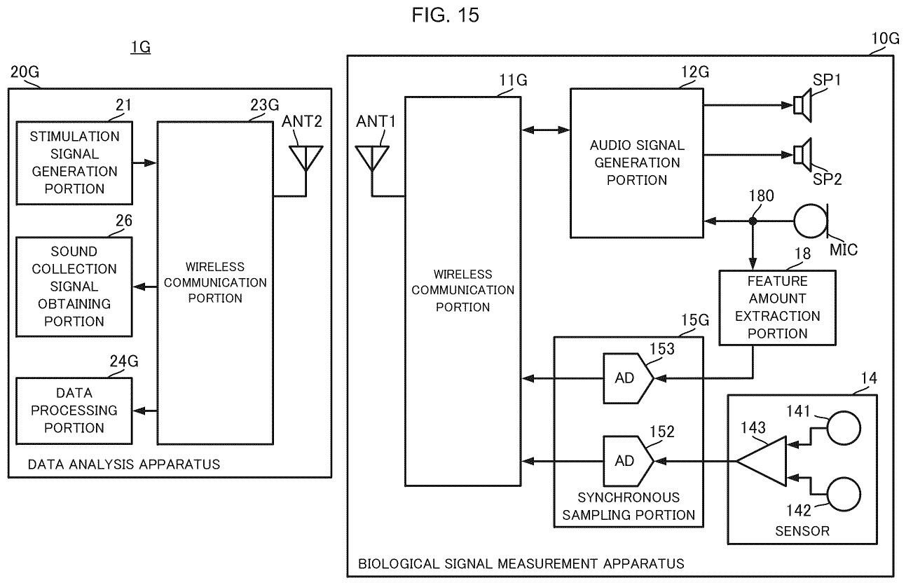

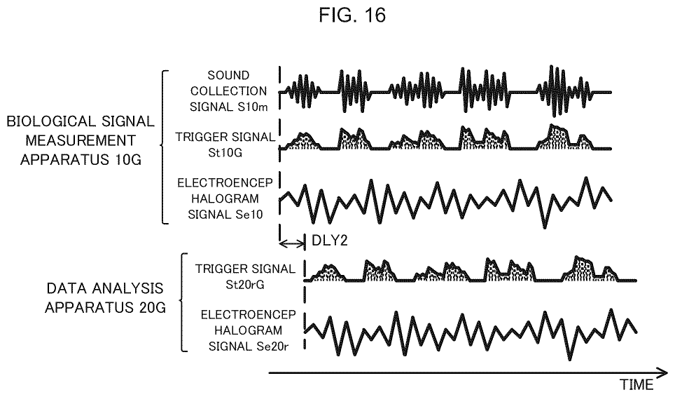

A biological signal measurement system according to an eighth embodiment will be described with reference to drawings. FIG. 15 is a functional block diagram showing one example of a configuration of the biological signal measurement system according to the eighth embodiment. FIG. 16 is a view showing a relationship on a time axis of each signal of the biological signal measurement system according to the eighth embodiment.

As shown in FIG. 15 and FIG. 16, the biological signal measurement system 1G according to the eighth embodiment is different from the biological signal measurement system 1D according to the fifth embodiment in that a biological signal measurement apparatus 10G and a data analysis apparatus 20G are provided and in that an electroencephalogram signal is synchronized with the sound collection signal. Other configurations of the biological signal measurement system 1G are the same as or similar to the configurations of the biological signal measurement system 1D, and a description of the same or similar configurations will be omitted.

The biological signal measurement system 1G includes a biological signal measurement apparatus 10G and a data analysis apparatus 20G.

The biological signal measurement apparatus 10G is different from the biological signal measurement system 1D in that a wireless communication portion 11G, an audio signal generation portion 12G, a synchronous sampling portion 15G, a sound collection signal obtaining portion 180, a feature amount extraction portion 18, and a microphone MIC are provided.

The microphone MIC collects a voice of a subject and generates a sound collection signal S10m, i.e., a captured audio signal. The microphone MIC outputs the sound collection signal S10m to the audio signal generation portion 12G.

The audio signal generation portion 12G AD converts the sound collection signal S10m and makes output to the wireless communication portion 11G. The wireless communication portion 11G sends the sound collection signal S10m to the wireless communication portion 23G of the data analysis apparatus 20G, through the antenna ANT1.

The sound collection signal obtaining portion 180 distributes the sound collection signal S10m to be output from the microphone MIC and makes output to the feature amount extraction portion 18.

The feature amount extraction portion 18 extracts a feature amount of the sound collection signal S10m and generates a trigger signal St10G. The feature amount extraction portion 18 outputs the trigger signal St10G to the synchronous sampling portion 15G.

The synchronous sampling portion 15G includes the sampling device 152 and a sampling device 153. The sampling device 152 and the sampling device 153 perform the AD sampling.

The sampling device 152 performs the digital sampling on the electroencephalogram signal Se10. The sampling device 153 performs the digital sampling on the trigger signal St10G. The sampling device 152 and the sampling device 153 synchronize and perform sampling.

The synchronous sampling portion 15G outputs the synchronously sampled trigger signal St10G and electroencephalogram signal Se10, to the wireless communication portion 11G.

The wireless communication portion 11G sends the synchronously sampled trigger signal St10G and electroencephalogram signal Se10, to the wireless communication portion 23G.

The data analysis apparatus 20G is different from the data analysis apparatus 20D in that the wireless communication portion 23G, a data processing portion 24G, and a sound collection signal obtaining portion 26 are provided.

The wireless communication portion 23G receives the sound collection signal S10m from the wireless communication portion 11G and makes output to the sound collection signal obtaining portion 26. In addition, the wireless communication portion 23G converts the data format of the trigger signal St10G received from the wireless communication portion 11G, and the electroencephalogram signal Se10, and makes output to the data processing portion 24G, as a trigger signal St20rG and the electroencephalogram signal Se20r.

The sound collection signal obtaining portion 26 performs the predetermined processing on the sound collection signal S10m. For example, the sound collection signal obtaining portion 26 stores the sound collection signal S10m in a storage medium. In addition, for example, the biological signal measurement apparatus 10G is a headset provided with the microphone MIC and the speakers SP1 and SP2, the data analysis apparatus 20G uses a PC (a personal computer), and, when communication for an audio conference or the like, of other PCs or the like is possible, the sound collection signal obtaining portion 26 converts the sound collection signal S10m into a format for communication with the other PCs and sends to the other PCs. It is to be noted that, in this embodiment, the sound collection signal obtaining portion 26, since directly giving no effect on processing of electroencephalogram analysis, is omissible.

The data processing portion 24G performs the predetermined data processing for the electroencephalogram measurement, based on the trigger signal St20rG and the electroencephalogram signal Se20r that are synchronized with each other.

By this configuration, the biological signal measurement system 1G is able to highly accurately measure the electroencephalogram signal to utterance of a subject while synchronizing with a generated sound.

It is to be noted that, in the present embodiment, the configurations (the stimulation signal generation portion 21 and the speaker SP1, the speaker SP2, or the like) that give a subject auditory stimulation are included, but are able to be omitted.

### Ninth Embodiment

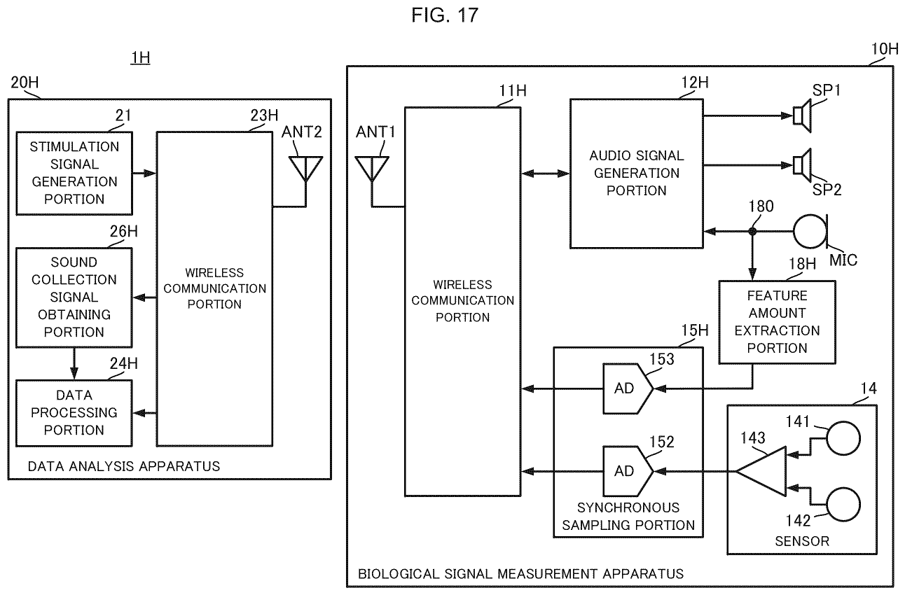

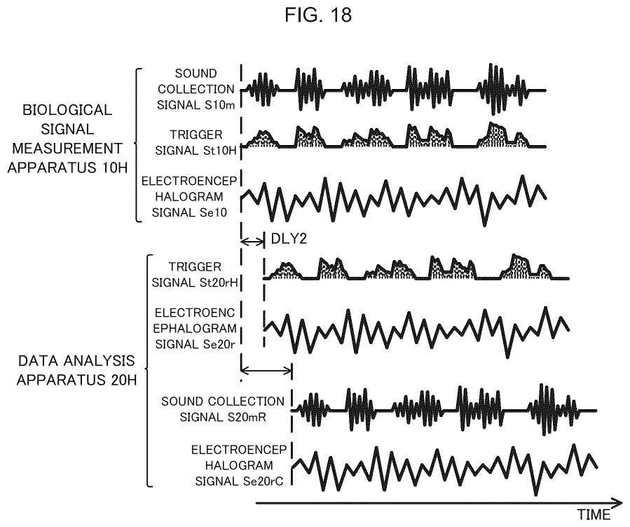

A biological signal measurement system according to a ninth embodiment will be described with reference to drawings. FIG. 17 is a functional block diagram showing one example of a configuration of the biological signal measurement system according to the ninth embodiment. FIG. 18 is a view showing a relationship on a time axis of each signal of the biological signal measurement system according to the ninth embodiment.

As shown in FIG. 17 and FIG. 18, the biological signal measurement system 1H according to the ninth embodiment is different from the biological signal measurement system 1G according to the eighth embodiment in that a biological signal measurement apparatus 10H and a data analysis apparatus 20H are provided and in that a sound collection signal received by the data analysis apparatus 20H and the electroencephalogram signal are synchronized with each other. Other configurations of the biological signal measurement system 1H are the same as or similar to the configurations of the biological signal measurement system 1G, and a description of the same or similar configurations will be omitted.

The biological signal measurement system 1H includes a biological signal measurement apparatus 10H and a data analysis apparatus 20H. The biological signal measurement apparatus 10H includes substantially the same configurations as the biological signal measurement apparatus 10G, and a wireless communication portion 11H, an audio signal generation portion 12H, a synchronous sampling portion 15H, and a feature amount extraction portion 18H each are substantially the same as the wireless communication portion 11G, the audio signal generation portion 12G, the synchronous sampling portion 15G, and the feature amount extraction portion 18G.

The data analysis apparatus 20H is different from the data analysis apparatus 20G in that a data processing portion 24H and a sound collection signal obtaining portion 26H are provided. It is to be noted that the wireless communication portion 23H of the data analysis apparatus 20H is substantially the same as the wireless communication portion 23G of the data analysis apparatus 20G.

The sound collection signal obtaining portion 26H extracts the feature amount of a sound collection signal S20mR obtained by converting the data format of the received sound collection signal S10m and generates an observation apparatus side trigger signal equivalent to a trigger signal St20rH in synchronization with the electroencephalogram signal Se20r. The sound collection signal obtaining portion 26H outputs the observation apparatus side trigger signal to the data processing portion 24H.

The data processing portion 24H includes a time relationship detection portion (a first time relationship detection portion). The time relationship detection portion of the data processing portion 24H detects a time relationship between the trigger signal St20rH and the sound collection signal S20mR, based on the trigger signal St20rH and the observation apparatus side trigger signal that have been received from the biological signal measurement apparatus 10H.

More specifically, the observation apparatus side trigger signal and the trigger signal St20rH are based on the feature amount of the sound collection signal S10m.

Accordingly, the data processing portion 24H compares waveforms of the trigger signal St20rH and the observation apparatus side trigger signal and detects the time relationship between the trigger signal St20rH and the sound collection signal S20mR.

Then, the sound collection signal S20mR and the observation apparatus side trigger signal are synchronized with each other. In addition, the electroencephalogram signal Se20r and the trigger signal St20rH are synchronized with each other.

Accordingly, the data processing portion 24H is able to synchronize the sound collection signal S20mR and the electroencephalogram signal Se20r, based on the detected time relationship between the sound collection signal S20mR and the trigger signal St20rH.

According to such a configuration, the data processing portion 24H is able to perform the predetermined data processing in a state in which the sound collection signal S20mR and the electroencephalogram signal Se20r are synchronized with each other. According to this, the biological signal measurement system 1H is able to more directly measure the electroencephalogram signal to the uttered voice of a subject.

### Tenth Embodiment

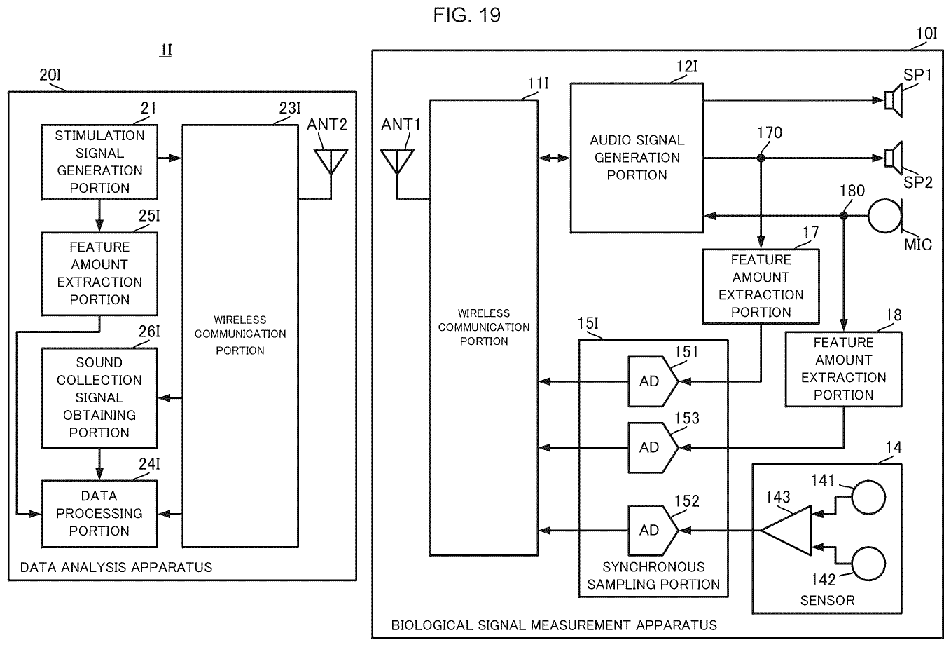

A biological signal measurement system according to a tenth embodiment will be described with reference to drawings. FIG. 19 is a functional block diagram showing one example of a configuration of the biological signal measurement system according to the tenth embodiment.

As shown in FIG. 19, the biological signal measurement system 1I according to the tenth embodiment is different from the biological signal measurement system 1H according to the ninth embodiment in that a biological signal measurement apparatus 10I and a data analysis apparatus 20I are provided and in that a trigger signal of an auditory stimulation signal, a sound collection signal received by the data analysis apparatus 20H, and an electroencephalogram signal are synchronized with one another. Other configurations of the biological signal measurement system 1I are the same as or similar to the configurations of the biological signal measurement system 1H, and a description of the same or similar configurations will be omitted.

The biological signal measurement system 1I includes a biological signal measurement apparatus 10I and a data analysis apparatus 20I.

The biological signal measurement apparatus 10I is different from the biological signal measurement apparatus 10H in that a synchronous sampling portion 15I, the feature amount extraction portion 17, and the distribution circuit 170 are provided. A wireless communication portion 11I, an audio signal generation portion 12I, and a feature amount extraction portion 18I each are substantially the same as the wireless communication portion 11H, the audio signal generation portion 12H, and the feature amount extraction portion 18H. The feature amount extraction portion 17 and the distribution circuit 170 each are substantially the same as the feature amount extraction portion 17 and distribution circuit 170 of the biological signal measurement apparatus 10D according to the fifth embodiment.

The synchronous sampling portion 15I includes a sampling device 151, a sampling device 152, and a sampling device 153. The sampling device 151 performs the AD sampling on the trigger signal based on the feature amount of the auditory stimulation signal. The sampling device 152 performs the AD sampling on the electroencephalogram signal. The sampling device 153 performs the AD sampling on the trigger signal based on the feature amount of the sound collection signal.

The sampling device 151, the sampling device 152, and the sampling device 153 perform the synchronous sampling. The synchronous sampling portion 15I outputs the trigger signal based on the feature amount of the auditory stimulation signal, the trigger signal based on the feature amount of the sound collection signal, and the electroencephalogram signal that have been synchronously sampled, to the wireless communication portion 11I.

The wireless communication portion 11I sends the trigger signal based on the feature amount of the auditory stimulation signal, the trigger signal based on the feature amount of the sound collection signal, and the electroencephalogram signal that have been synchronously sampled, to a wireless communication portion 23I.

The data analysis apparatus 20I is different from the data analysis apparatus 20H in that a data processing portion 24I and a feature amount extraction portion 25I are provided. The wireless communication portion 23I and the sound collection signal obtaining portion 26I each are substantially the same as the wireless communication portion 23H and the sound collection signal obtaining portion 26H.

The feature amount extraction portion 25I is substantially the same as the feature amount extraction portion 25 according to the sixth embodiment.

The data processing portion 24I performs the predetermined data processing, based on the trigger signal based on the feature amount of the auditory stimulation signal, the trigger signal based on the feature amount of the sound collection signal, and the electroencephalogram signal that have been synchronously sampled.

According to this, the data processing portion 24I is able to highly accurately perform analysis of brain waves to be generated to the auditory stimulation and utterance of a subject.

Further, the data processing portion 24I including the above-described configuration is able to synchronize the auditory stimulation signal, the sound collection signal, and the electroencephalogram signal and perform the predetermined data processing. According to this, the data processing portion 24I is able to more directly perform the analysis of brain waves to the auditory stimulation and utterance of a subject and more highly accurately perform the analysis of brain waves.

### Advantageous Effects

According to the present disclosure, synchronization between a stimulation signal and a biological signal is able to be reliably performed even when communication delay occurs between a device that generates a stimulation signal and a device that obtains a biological signal.

The present disclosure provides a specific technical improvement to the functioning of biological signal measurement systems operating over wireless networks that experience variable latency. Unlike conventional approaches that rely on maintaining precise internal clock synchronization between a measurement device and an analysis device—which is prone to error due to unpredictable transmission delays (e.g., DLY1 and DLY2)—the present configuration utilizes synchronous sampling circuitry to structurally pair a locally obtained trigger signal with the biological signal at the point of measurement. By generating a data set where the trigger and biological signals are already aligned prior to wireless transmission, the system eliminates the dependency on constant transmission speeds or synchronized system clocks. This configuration overcomes a technical problem specifically arising in the realm of wireless biological monitoring, thereby improving the accuracy and reliability of the data analysis apparatus without requiring increased computational resources for clock synchronization.

<1> A biological signal measurement system including a biological signal measurement apparatus including a sensor that measures a biological signal based on reaction of a subject to stimulation, and a first wireless communication portion, and a data analysis apparatus including a data processing portion that performs predetermined data processing to the biological signal, and a second wireless communication portion that performs data communication with the first wireless communication portion, the data analysis apparatus including a stimulation signal generation portion that generates a stimulation signal for giving stimulation, the biological signal measurement apparatus including a trigger signal obtaining portion that obtains a trigger signal corresponding to the stimulation signal, and a synchronous sampling portion that synchronizes and samples the trigger signal and the biological signal, the second wireless communication portion sending the stimulation signal to the first wireless communication portion, the first wireless communication portion receiving the stimulation signal from the second wireless communication portion, the first wireless communication portion sending the trigger signal and the biological signal that have been sampled by the synchronous sampling portion to the second wireless communication portion, the second wireless communication portion receiving the trigger signal and the biological signal that have been sampled by the synchronous sampling portion, from the second wireless communication portion, and the data processing portion performing the predetermined data processing, based on the received biological signal and trigger signal.

<2> The biological signal measurement system according to <1>in which the stimulation signal generation portion generates a first pulse signal of a pulse waveform repeated in a predetermined cycle, together with the stimulation signal, the second wireless communication portion sends the first pulse signal together with the stimulation signal to the first wireless communication portion, and the trigger signal obtaining portion obtains the first pulse signal as the trigger signal.

<3> The biological signal measurement system according to <1> in which the data analysis apparatus includes a first feature amount extraction portion that extracts a feature amount of the stimulation signal, the second wireless communication portion sends the feature amount together with the stimulation signal to the first wireless communication portion, and the trigger signal obtaining portion obtains the feature amount as the trigger signal.

<4> The biological signal measurement system according to <1> in which the data analysis apparatus includes a first feature amount extraction portion that extracts a feature amount of the stimulation signal, the second wireless communication portion sends the feature amount together with the stimulation signal to the first wireless communication portion, and

the trigger signal obtaining portion generates a second pulse signal of a pulse waveform binarized by whether or not a predetermined condition is satisfied out of the feature amount arranged in time series, and obtains the second pulse signal as the trigger signal.

<5> The biological signal measurement system according to <1> in which the data analysis apparatus includes a first feature amount extraction portion that extracts a feature amount of the stimulation signal, and a trigger signal generation portion that generates a second pulse signal of a pulse waveform binarized by whether or not a predetermined condition is satisfied out of the feature amount and generates the second pulse signal as the trigger signal, the second wireless communication portion sends the second pulse signal together with the stimulation signal to the first wireless communication portion, and the trigger signal obtaining portion obtains the second pulse signal as the trigger signal.

<6> The biological signal measurement system according to <1> in which the biological signal measurement apparatus includes a second feature amount extraction portion that extracts a feature amount of the stimulation signal, and the trigger signal obtaining portion obtains the feature amount as the trigger signal.

<7> The biological signal measurement system according to <1> in which the biological signal measurement apparatus includes

a second feature amount extraction portion that extracts a feature amount of the stimulation signal, and the trigger signal obtaining portion generates a second pulse signal of a pulse waveform binarized by whether or not a predetermined condition is satisfied out of the feature amount, and obtains the second pulse signal as the trigger signal.

<8>T he biological signal measurement system according to any of <3> to <7> in which the data analysis apparatus includes a delay time estimation portion that estimates delay time of the stimulation signal and the feature amount from a time difference between the feature amount sent from the second wireless communication portion to the first wireless communication portion and the feature amount or the trigger signal that has been sampled by the synchronous sampling portion and that the second wireless communication portion has received from the first wireless communication portion.

<9> The biological signal measurement system according to any of <1> to <8> in which the biological signal measurement apparatus includes a first output portion and a second output portion each connected to the first wireless communication portion, and a switch circuit that switches a connection relationship between the first output portion and the second output portion, and the first wireless communication portion, and the switch circuit transmits the stimulation signal from the first wireless communication portion to the first output portion and the second output portion, and switches between a first connection mode of transmitting the trigger signal from the first wireless communication portion to the synchronous sampling portion and a second connection mode of transmitting a different stimulation signal from the first wireless communication portion to the first output portion and the second output portion.

<10> The biological signal measurement system according to any of <1> to <9> in which the stimulation is auditory stimulation, and the biological signal is an electroencephalogram signal.

<11> A biological signal measurement system including a biological signal measurement apparatus including a microphone that collects a sound that a subject utters and generates a sound collection signal, a sensor that measures a biological signal of the subject to the sound, and a first wireless communication portion, a data analysis apparatus including a data processing portion that performs predetermined data processing to the biological signal, and a second wireless communication portion that performs data communication with the first wireless communication portion, the biological signal measurement apparatus including a feature amount extraction portion that extracts a feature amount of the sound collection signal, a first trigger signal obtaining portion that obtains a first trigger signal based on the feature amount, and a synchronous sampling portion that synchronizes and samples the first trigger signal and the biological signal, the first wireless communication portion sending the first trigger signal and the biological signal synchronized and sampled with the first trigger signal to the second wireless communication portion, and the data processing portion of the data analysis apparatus performing the predetermined data processing, based on the received biological signal and first trigger signal.

<12> The biological signal measurement system according to <11> in which the first trigger signal obtaining portion obtains the feature amount as the first trigger signal.

<13> The biological signal measurement system according to <11> in which the first trigger signal obtaining portion generates a pulse signal of a pulse waveform binarized by whether or not a predetermined condition is satisfied out of the feature amount, and obtains the pulse signal as the first trigger signal.

<14> The biological signal measurement system according to any of <11> to <13> in which the first wireless communication portion of the biological signal measurement apparatus sends the sound collection signal to the second wireless communication portion, the data analysis apparatus includes a first time relationship detection portion that detects a time relationship between the sound collection signal and the first trigger signal, and a synchronous processing portion that performs synchronization between the sound collection signal and the biological signal from the time relationship between the sound collection signal and the first trigger signal, and the data processing portion performs the predetermined data processing by using the synchronized sound collection signal and biological signal.

<15> The biological signal measurement system according to any of <11> to <14> in which the data analysis apparatus includes a stimulation signal generation portion that generates a stimulation signal for giving stimulation, the biological signal measurement apparatus includes a second trigger signal obtaining portion that obtains a second trigger signal corresponding to the stimulation signal, and the synchronous sampling portion that synchronizes and samples the first trigger signal, the second trigger signal, and the biological signal, the second wireless communication portion sends the stimulation signal to the first wireless communication portion, the first wireless communication portion sends the synchronously sampled first trigger signal, second trigger signal, and biological signal, to the second wireless communication portion, and the data processing portion of the data analysis apparatus performs the predetermined data processing by using the received biological signal, first trigger signal, and second trigger signal.

<16> The biological signal measurement system according to <15> in which the data analysis apparatus includes a second time relationship detection portion that detects a time relationship between the stimulation signal and the second trigger signal, and a synchronous processing portion that performs synchronization between the stimulation signal and the biological signal from the time relationship between the stimulation signal and the second trigger signal, and the data processing portion performs the predetermined data processing by using the synchronized stimulation signal, sound collection signal, and biological signal.

<17> The biological signal measurement system according to any of <11> to <16> in which the biological signal is an electroencephalogram signal.

<18> The biological signal measurement system according to <15> or <16> in which the stimulation is auditory stimulation, and the biological signal is an electroencephalogram signal.

<19> A biological signal measurement apparatus including a trigger signal obtaining portion that obtains a trigger signal corresponding to a stimulation signal from outside, a sensor that measures a biological signal based on reaction of the subject to the stimulation signal, a synchronous sampling portion that synchronizes and samples the trigger signal and the biological signal, and a wireless communication portion that performs transmission and reception of data with a data analysis apparatus, the wireless communication portion receiving the trigger signal from the data analysis apparatus, sends to the trigger signal obtaining portion, and sending the trigger signal and the biological signal that have been sampled by the synchronous sampling portion, to the data analysis apparatus.

<20> A biological signal measurement apparatus including a microphone that collects a sound that a subject utters and generates a sound collection signal, a sensor that measures a biological signal of the subject to the sound, and a feature amount extraction portion that extracts a feature amount of the sound collection signal, a trigger signal obtaining portion that obtains a trigger signal based on the feature amount, a synchronous sampling portion that synchronizes and samples the trigger signal and the biological signal, and a wireless communication portion that sends the trigger signal and the biological signal synchronized and sampled with the trigger signal, to a data analysis apparatus.

<21> A data analysis apparatus including a stimulation signal generation portion that generates a stimulation signal for giving the stimulation, a wireless communication portion that receives a biological signal based on reaction of a subject to the stimulation, and a trigger signal synchronously sampled with the biological signal and corresponding to the stimulation signal, and a data processing portion that performs predetermined data processing with respect to biological reaction corresponding to the biological signal, based on the received biological signal and trigger signal.

<22> A biological signal processing method in which a biological signal measurement apparatus measures a biological signal based on reaction of a subject to stimulation, a data analysis apparatus performs predetermined data processing to the biological signal, the data analysis apparatus generates a stimulation signal for giving the stimulation, the stimulation signal is sent from the data analysis apparatus to the biological signal measurement apparatus, the biological signal measurement apparatus obtains a trigger signal corresponding to the received stimulation signal and synchronizes and samples the trigger signal and the biological signal, the synchronously sampled trigger signal and biological signal are sent from the biological signal measurement apparatus to the data analysis apparatus, and the data analysis apparatus performs the predetermined data processing, based on the received biological signal and trigger signal.

<23> A biological signal processing method in which a biological signal measurement apparatus collects a sound that a subject utters and generates a sound collection signal, and measures a biological signal of the subject to the sound, a data analysis apparatus performs predetermined data processing to the biological signal, the biological signal measurement apparatus extracts a feature amount of the sound collection signal, obtains a trigger signal based on the feature amount and synchronizes and samples the trigger signal and the biological signal, the trigger signal and the biological signal synchronized and sampled with the trigger signal are sent from the biological signal measurement apparatus to the data analysis apparatus, and the data analysis apparatus performs the predetermined data processing, based on the received biological signal and trigger signal.

<24> A biological signal measurement program that causes a biological signal measurement apparatus to execute: processing to obtain a trigger signal corresponding to a stimulation signal from outside, processing to measure a biological signal based on reaction of a subject to stimulation by the stimulation signal, processing to synchronize and sample the trigger signal and the biological signal, and processing to send the synchronously sampled trigger signal and biological signal.

<25> A biological signal measurement program that causes a biological signal measurement apparatus to execute: processing to collect a sound that a subject utters and generates a sound collection signal, processing to measure a biological signal to the sound, processing to extract a feature amount of the sound collection signal, processing to obtain a trigger signal based on the feature amount, processing to synchronize and sample the trigger signal and the biological signal, and processing to send the trigger signal and the biological signal synchronized and sampled with the trigger signal.

<26> A data analysis program that causes a data analysis apparatus to execute: processing to generate a stimulation signal for giving the stimulation, processing to receive a biological signal based on reaction of a subject to the stimulation, and a trigger signal synchronously sampled with the biological signal and corresponding to the stimulation signal, and processing to perform predetermined data processing with respect to biological reaction corresponding to the biological signal, based on the received biological signal and trigger signal.

### REFERENCE SIGNS LIST

1, 1A, 1B, 1C, 1D, 1E, 1F, 1G, 1H, 1I: biological signal measurement system
10, 10B, 10C, 10D, 10E, 10F, 10G, 10H, 10I: biological signal measurement apparatus
11, 11G, 11H, 11I: wireless communication portion
12, 12G, 12H, 12I: audio signal generation portion
13 13B: trigger signal obtaining portion
14: sensor
15, 15G, 15H, 15I: synchronous sampling portion
17, 18, 18H: feature amount extraction portion
19: switch circuit
20, 20A, 20B, 20D, 20E, 20F, 20G, 20H, 20I: data analysis apparatus
21 21D: stimulation signal generation portion
22: trigger signal generation portion
23, 23G, 23H, 23I: wireless communication portion
24, 24A, 24B, 24D, 24E, 24F, 24G, 24H, 24I: data processing portion
25 25I: feature amount extraction portion
26, 26H, 26I: sound collection signal obtaining portion
141, 142: electrode
143: amplifier
151, 152, 153: sampling device
170: distribution circuit
180: sound collection signal obtaining portion
ANT1, ANT2: antenna
DLY1, DLY12, DLY2: delay time
MIC: microphone
S10, S10L, S10R: auditory stimulation signal
S10m, S20mR: sound collection signal
S20t, S20tL, S20tR: auditory stimulation signal
Se10, Se20r: electroencephalogram signal
SP1, SP2: speaker
St10, St10A, St10B, St10D, St10G, St20F, St20r, St20rA,
St20rD, St20rE, St20rF, St20rG, St20rH, St20t, 20tA, St20tE: trigger signal
SW1, SW2: switch element
THp: threshold value

1. A biological signal measurement system comprising:
a biological signal measurement apparatus including a sensor that measures a biological signal based on reaction of a subject to stimulation, and a first wireless communication circuit; and
a data analysis apparatus including data processing circuitry configured to perform predetermined data processing to the biological signal, and second wireless communication circuitry configured to perform data communication with the first wireless communication circuitry, wherein:
the data analysis apparatus includes a stimulation signal generation circuitry that generates a stimulation signal for giving the stimulation;
the biological signal measurement apparatus includes: a trigger signal acquisition circuitry configured to obtain a trigger signal corresponding to the stimulation signal; and
a synchronous sampling circuitry configured to synchronize and sample the trigger signal and the biological signal to generate a synchronously sampled data set;
the second wireless communication circuitry is configured to transmit the stimulation signal to the first wireless communication circuitry;
the first wireless communication circuitry is configured to receive the stimulation signal from the second wireless communication circuitry;
the first wireless communication circuitry is configured to transmit the synchronously sampled data set to the second wireless communication circuitry;
the second wireless communication circuitry receives the trigger signal and the biological signal from the first wireless communication circuitry; and
the data processing circuitry performs the predetermined data processing, based on the received biological signal and trigger signal.

2. The biological signal measurement system according to claim 1, wherein:
the stimulation signal generation circuitry is configured to generate a first pulse signal of a pulse waveform repeated in a predetermined cycle, together with the stimulation signal;
the second wireless communication is configured to send the first pulse signal together with the stimulation signal to the first wireless communication circuitry; and
the trigger signal acquisition circuitry is configured to obtain the first pulse signal as the trigger signal.

3. The biological signal measurement system according to claim 1, wherein:
the data processing circuitry is configured to extract a feature amount of the stimulation signal;
the second wireless communication circuitry is configured to send the feature amount together with the stimulation signal to the first wireless communication portion; and
the trigger signal acquisition circuitry is configured to obtain the feature amount as the trigger signal.

4. The biological signal measurement system according to claim 1, wherein:
the data processing circuitry is configured to extract a feature amount of the stimulation signal;
the second wireless communication circuitry is configured to send the feature amount together with the stimulation signal to the first wireless communication circuitry; and
the trigger signal acquisition circuitry is configured to generate a second pulse signal of a pulse waveform binarized by whether or not a predetermined condition is satisfied out of the feature amount arranged in time series, and obtain the second pulse signal as the trigger signal.

5. The biological signal measurement system according to claim 1, wherein:
the biological signal measurement apparatus includes:
a feature amount extraction circuitry configured to extract a feature amount of the stimulation signal; and
a trigger signal acquisition circuitry configured to generate a second pulse signal of a pulse waveform binarized by whether or not a predetermined condition is satisfied out of the feature amount arranged in time series and generate the second pulse signal as the trigger signal;
the second wireless communication circuitry is configured to send the second pulse signal together with the stimulation signal to the first wireless communication circuitry; and
the trigger signal acquisition circuitry is configured to obtain the second pulse signal as the trigger signal.

6. The biological signal measurement system according to claim 1, wherein:
the biological signal measurement apparatus includes a feature amount extraction circuitry configured to extract a feature amount of the stimulation signal; and
the trigger signal acquisition circuitry is configured to obtain the feature amount as the trigger signal.

7. The biological signal measurement system according to claim 1, wherein:
the biological signal measurement apparatus includes a second feature amount extraction circuitry configured to extract a feature amount of the stimulation signal; and
the trigger signal acquisition circuitry configured to generate a second pulse signal of a pulse waveform binarized by whether or not a predetermined condition is satisfied out of the feature amount, and obtain the second pulse signal as the trigger signal.

8. The biological signal measurement system according to claim 3, wherein the data analysis apparatus includes a delay time estimation circuitry configured to estimate delay time of the stimulation signal and the feature amount from a time difference between the feature amount sent from the second wireless communication circuitry to the first wireless communication circuitry and the feature amount or the trigger signal that has been sampled by the synchronous sampling circuitry and that the second wireless communication circuitry has received from the first wireless communication circuitry.

9. The biological signal measurement system according to claim 1, wherein:
the biological signal measurement apparatus includes:
a first output interface and a second output interface each connected to the first wireless communication circuitry; and
a switch circuitry configured to
switch between:
a first connection mode that transmits the trigger signal from the first wireless communication portion to the synchronous sampling circuitry; and
a second connection mode that transmits a different stimulation signal from the first wireless communication circuitry to the first output interface and the second output interface.

10. The biological signal measurement system according to claim 1, wherein:
the stimulation is auditory stimulation; and
the biological signal is an electroencephalogram signal.

11. The biological signal measurement system according to claim 1, wherein:
the biological signal measurement apparatus further includes
a microphone configured to collect a sound uttered by the subject and generate a sound collection signal; and
a feature amount extraction circuitry configured to extract a feature amount of the sound collection signal;
wherein the trigger signal acquisition circuitry is further configured to obtain a second trigger signal based on the feature amount of the sound collection signal.

12. The biological signal measurement system according to claim 11, wherein the synchronous sampling circuitry is configured to synchronize and sample the trigger signal corresponding to the stimulation signal, the second trigger signal corresponding to the sound collection signal, and the biological signal.

13. A biological signal measurement apparatus comprising:
a first wireless communication circuitry configured to receive a stimulation signal from a data analysis apparatus;
a trigger signal acquisition circuitry configured to obtain a trigger signal corresponding to the stimulation signal;
a sensor configured to measure a biological signal based on a reaction of a subject to the stimulation signal;
a synchronous sampling circuitry configured to synchronize and sample the trigger signal and the biological signal to generate a sampled data set; and
a processor circuitry configured to control the first wireless communication circuitry to transmit the sampled data set to the data analysis apparatus.

14. The biological signal measurement apparatus according to claim 13, further comprising:
a microphone configured to collect a sound uttered by the subject and generate a sound collection signal; and
a feature amount extraction circuitry configured to extract a feature amount of the sound collection signal; wherein the trigger signal acquisition circuitry is further configured to obtain a second trigger signal based on the feature amount of the sound collection signal.

15. The biological signal measurement apparatus according to claim 14, wherein the trigger signal acquisition circuitry is configured to obtain the feature amount as the second trigger signal.

16. The biological signal measurement apparatus according to claim 14, wherein the trigger signal acquisition circuitry is configured to generate a pulse signal of a pulse waveform binarized by whether or not a predetermined condition is satisfied out of the feature amount, and obtain the pulse signal as the second trigger signal.

17. The biological signal measurement apparatus according to claim 13, wherein:
the synchronous sampling circuitry is configured to synchronize and sample the trigger signal corresponding to the stimulation signal, the second trigger signal corresponding to the sound collection signal, and the biological signal.

18. The biological signal measurement apparatus according to claim 13, wherein the biological signal is an electroencephalogram signal.

19. A data analysis apparatus comprising:
a stimulation signal generation circuitry configured to generate a stimulation signal for giving stimulation to a subject;
a wireless communication circuitry configured to:
transmit the stimulation signal to a biological signal measurement apparatus; and
receive, from the biological signal measurement apparatus, a biological signal and a trigger signal, the biological signal based on reaction of the subject to the stimulation, and the trigger signal synchronously sampled with the biological signal by the biological signal measurement apparatus; and
a data processing circuitry configured to perform predetermined data processing regarding a biological reaction based on the received biological signal and the trigger signal.

20. The data analysis apparatus according to claim 19, wherein the data processing circuitry is further configured to:
detect a time relationship between the stimulation signal generated by the stimulation signal generation circuitry and the trigger signal received from the biological signal measurement apparatus; and
synchronize the stimulation signal and the biological signal based on the detected time relationship.
